(19) United States
(12) Patent Application Publication
(10) Pub. No.: US 2013/0344606 A1
(43) Pub. Date: Dec. 26, 2013

(54) Heparin Concentration and Heparin Response Imbalance Determination Method Within a Fluid Containing Heparin

(71) Applicant: Viscell, LLC, Boulder, CO (US)

(72) Inventors: Jon Harry HENDERSON, Boulder, CO (US); Barbara Ann DeBIASE, Boulder, CO (US)

(21) Appl. No.: 13/912,620

(22) Filed: Jun. 7, 2013

Related U.S. Application Data
(60) Provisional application No. 61/658,139, filed on Jun. 11, 2012.

(51) Int. Cl. G01N 33/86

(52) U.S. Cl. CPC G01N 33/86
USPC 436/69

(57) ABSTRACT

Provided herein are various methods for determining heparin concentration or heparin response imbalance in native whole blood, citrated whole blood, or plasma by measuring two parameters that characterize each phase of a two-phase coagulation response, such as a time period until clot formation initiation and a post-initiation clot formation.

### CROSS-REFERENCE TO RELATED APPLICATION

This application claims benefit to U.S. Provisional Patent 61/658,139 filed Jun. 11, 2012, which is specifically incorporated by reference.

### BACKGROUND OF THE INVENTION

Blood has the ability to change from a liquid into a clot. This physiological process, coagulation, is complex and involves multiple chemical reactions that progress sequentially. The coagulation process is typically quantified by adding an activator to a blood sample and measuring the time period between activation and initial clot formation. While various activators are used to characterize different aspects of coagulation, these tests share a common testing methodology of measuring a time period and using this time result to characterize coagulation performance.

Most historical devices for characterizing clotting involve chemically activating the clotting process, automatically detecting the resulting clot, and timing the process until the clot is detected. Herein, this class of instruments is referred to as Clot Timers.

While Clot Timers can detect a clot, more advanced analyzers incorporate instrumentation that can differentiate physical properties of a developing clot. Such devices rely on varied instrumentation approaches to characterize the clot including: measuring changes in optical transmission through a plasma, measuring color changes within plasma samples treated with chromogenic materials, measuring the strength of the clot (thromboelastography), measuring viscoelastic changes in the clot (Sonoclot™) measuring thrombin levels dynamically during clotting, or measuring rheology changes within the clotting sample using ultrasonic methods. Herein this class of instruments are referred to as Global Hemostasis Monitors.

Modeling the coagulation process from the perspective of a Global Hemostasis Monitor is not straightforward because each device incorporates different terminology to characterize the clot integrity and report test results. By using a model that focuses on coagulation rather than instrumentation, a common methodology can be used to characterize results from any of the Global Hemostasis Monitor instruments. Herein, the processes of coagulation reactions and clot development are combined within a simplified Two Phase Clotting Model illustrated by FIG. 1. This model consists of a graph; the X axis is time, and the Y axis is a characterization of the physical state of the clot. The physical aspect of a clot is characterized with a generic term, Clot Integrity, a quantitative assessment of the physical nature of the clot. Prior to any clot formation, Clot Integrity is zero. Clot Integrity increases during clot formation. Weak clots have lower Clot Integrity than strong clots. Plotted on this graph is a curve that tracks Clot Integrity measurements versus time. A simplified characterization of the Clot Integrity curve splits the clotting process into two phases: a first phase where coagulation reactions are developing prior to any clot formation, and a second phase where clot formation develops, i.e., Clot Integrity increases. These phases are herein referred to as a Coagulation Reaction Phase and a Clot Formation Phase; see FIG. 1. For numerical analysis, the Coagulation Reaction Phase is characterized with a Reaction Time (T), and the Clot Formation Phase is characterized with a Formation Rate (R), the rate of change in Clot Integrity. The Two Phase Clotting Model extends quantitative assessment of coagulation from measurement of a single time result to measurement of two results, T and R.

Heparin is a naturally occurring polysaccharide. When it binds with antithrombin, a protein within blood, the heparin antithrombin complex alters many clotting factors and reactions. Heparin affects the clot formation process in multiple ways. Two observable effects on clot development are: higher heparin concentrations prolong both the Coagulation Reaction Phase and the Clot Formation Phase. These effects are illustrated using the output from a Sonoclot™ Analyzer in FIG. 2. The same blood sample is run with different heparin concentrations. From a quantitative perspective heparin increases T and reduces R. T varies with heparin in an approximately linear rate; see FIG. 3. The change in R is non-linear; see FIG. 4. R decreases with increasing heparin concentrations. R is very sensitive at low heparin concentrations and becomes less sensitive at higher heparin concentrations.

Heparin is used clinically as an anticoagulant to prevent and/or treat blood clots. It is available in conventional unfractionated and newer low molecular weight forms. Proper dosing of heparin during treatment improves patient outcomes since under-administration elevates the risk of forming unwanted blood clots and over-administration elevates the risk of bleeding. Heparin is metabolized. In order to maintain desired therapeutic heparin concentrations, heparin must be re-administrated periodically. Since heparin metabolism varies considerably among patients, accurate heparin dosing requires patient heparin monitoring to guide heparin re-administration.

Heparin administration is typically managed with a Clot Timer instrument. T is prolonged in the presence of heparin. A target time is established for managing the heparin concentration. When T is less than the target time, additional heparin is administered. Several tests commonly used for managing heparin include an Activated Clotting Time (ACT) and an activated partial thromboplastin time (APTT). Both ACT and APTT results are times and measure the T result for the Two Phase Coagulation Model although actual test result ranges differ due to the use of different activators and sample handling procedures. A limitation of heparin monitoring tests based on measuring only T is imprecision to actual heparin concentration due to multiple sources of measurement variance. Significant sources of variance include patient to patient variance in T when no heparin is present and patient to patient variance in response to increasing heparin concentrations.

The patient to patient variance in the T when no heparin is present can be corrected if either T is measured prior to heparin administration or if T is measured on a sample with the heparin neutralized. For convenience, both aspects are broadly included in the term sample without heparin. With this testing, two T results are calculated: one on the sample without heparin and the other on the sample with heparin. The difference between T in the sample containing heparin and T in the sample without heparin (ΔT) is used to establish a numerical relationship to heparin concentration.

While heparin neutralization testing with the ΔT result substantially eliminates patient to patient variance when the heparin concentration is zero, ΔT does not correct patient to patient variance in response to increasing heparin concentrations. Both the T and the ΔT results are affected by the patient to patient variance of T to increasing heparin concentrations.

Heparin can be neutralized from a blood sample prior to testing with several different reagents including polybrene, protamine sulfate, or heparinase. The most useful reagent for calculating a ΔT result is heparinase because it neutralizes heparin without altering the coagulation and clot development process whereas polybrene and protamine sulfate do alter the coagulation and clot development process. Heparinase testing is useful in identifying small amounts of heparin since the ΔT result significantly eliminates patient to patient variability in the T result at low heparin concentrations.

However, heparinase testing as introduced by Folkman in U.S. Pat. No. 4,795,703 has not achieved significant market success in managing heparin administration. An explanation for this lack of market success is that ΔT accuracy improvements are only significant at low heparin concentrations. Also, heparinase testing is more expensive and running two tests is more complicated than running a single test on a blood sample.

The overall performance for using Clot Timer instruments and a T result for managing heparin therapy is recognized as imprecise, but Clot Timer instruments nevertheless dominate the point of care heparin management market. During heparin therapy, some patients still bleed, blood component circuits occasionally occlude with blood clots, some patients still develop blood clots in repaired vessels, and thrombosis is a risk that can cause organ damage, stroke or death. Improved point of care precision of heparin management assessment, therefore, plays an important role in achieving improved patient outcomes.

Global Hemostasis Monitors have been available for many years but have only found limited use within heparin management. Duplicate testing with and without heparinase has been used both to evaluate hemostasis performance of the heparin neutralized sample and as a means to detect small amounts of heparin within a blood sample by observing results between the sample containing heparin and the heparin neutralized sample. None of the Global Hemostasis Monitors have used T and R equivalent results that have been combined into an improved heparin concentration estimate for the user.

Researchers, including Babski et al. (J Vet Intern Med, Volume 26, Issue 3, 2012, pp 631-638), have published results that show both the Sonoclot ACT, a result that quantifies T, and the Sonoclot CR, a result that quantifies R, correlate well to anti-Xa activity (and heparin) and a multiple linear regression using both ACT and R results provided higher statistical correlation than either result separately. The linear regression techniques used by Babski was one of many statistical analyses performed on a dataset. There was no effort nor intent by Babski to incorporate both the ACT and CR results into a functional device that combined multiple heparin concentration assessment results into single heparin concentration result. It is important to note that a multivariable linear regression is based on the assumption that T and R are independent variables and the heparin concentration is a function of T and R. This approach did show slight improvement in statistical correlation, but is flawed because T and R are not independent variables. Rather, both T and R are dependent on heparin concentration. Heparin concentration is the independent variable being assessed by the separate results, T and R, that change in response to heparin concentration. Properly relating T and R results into an accurate and useful tool for heparin concentration estimation requires a more comprehensive analysis of non linearities and/or variances within the defining relationships between test results and actual heparin concentrations.

A more accurate measurement of heparin can be obtained using an anti-Xa laboratory test. This test is calibrated to report test results as a heparin concentration in plasma rather than a time, and the results are a more accurate measurement of heparin than what can be achieved with a Clot Timer instrument. The anti-Xa test is considered the “gold standard” for heparin concentration measurement precision. However, even this test has patient to patient variations in results, is expensive, and is not available as a point of care test for immediate patient management needs. The anti-Xa offers precision but does not meet user requirements for convenience, cost, and processing time.

Yet another means for measuring heparin concentration is protamine titration. Protamine sulfate is used to stoichiometrically neutralize predetermined amounts of heparin from multiple aliquots of blood containing an unknown heparin concentration. This method requires two processing steps. First a fixed amount of protamine is added to a sample containing heparin, and second, a clotting analysis is performed on the sample with a Clot Timer device. Varying amounts of protamine sulfate are added to separate aliquots of a test sample and the resulting Reaction Times are analyzed to calculate a heparin concentration measurement. This technique is complicated and expensive. Further, protamine titration provides limited accuracy since individual titration points are discrete which limits resolution. Also, test cartridges typically only cover a limited heparin range; if heparin concentrations are above or below the test range, test results are inconclusive. Protamine sulfate titration is used far less frequently than Clot Timers for heparin management.

U.S. Pat. No. 7,699,966 describes a point of care method for determining heparin concentration in blood using cartridges that include protamine ion sensitive electrodes and reference electrodes to perform a protamine titration. That method is a new variation of protamine titration and little is known about cost or performance.

Heparin alters multiple coagulation reactions. Some patients have abnormal response to heparin. If the intended anticoagulant effect is not achieved after heparin administration, the patient is at elevated risk for thrombosis or bleeding. Currently, heparin resistance is identified when the desired increase in a clotting time is not achieved after administration of a measured heparin dose. This approach only considers heparin resistance that fails to prolong the clotting time. Heparin resistance can also occur when the clotting time is prolonged but Clot Formation Phase does not respond to heparin normally, and a clot develops faster than expected after initial clot formation. This type of heparin resistance is associated with abnormal performance during the Clot Formation Phase; currently, this type of heparin resistance identification is not practiced clinically.

### SUMMARY OF THE INVENTION

Provided herein are methods and systems that efficiently, reliably, and precisely monitor a heparin parameter from a sample, including heparin concentration and/or abnormal response to heparin that cannot be achieved by conventional systems. In particular, the use of two separate parameters, each related to a different aspect of the two-phase coagulation model, provides a basis for improved characterization of heparin and response to heparin by an individual.

In an embodiment, the invention is a method for determining a heparin parameter in a fluid sample that may include heparin by providing a fluid sample from an individual. A first parameter and a second parameter are measured from the fluid sample, wherein the first and second parameter each vary with heparin concentration in the fluid sample. The first parameter is used to calculate a first intermediate result, and the second parameter the second intermediate result. The first and said second intermediate results are combined to determine the heparin parameter. In an aspect, the heparin parameter is heparin concentration or heparin response imbalance. In an aspect, the heparin parameter is both heparin concentration and heparin response imbalance, such as providing a heparin concentration and notification that the individual's response to heparin falls within a normative range. Alternatively, a heparin concentration may be reported along with notification that the individual's response to heparin falls outside a normative range, inviting further investigation or treatment modification such as discontinuation of heparin therapy, treatment with other compounds and/or modification of heparin dosage.

The methods provided herein are useful in determining heparin concentration of unfractionated heparin or low molecular weight heparin. As used herein, low molecular weight heparin refers to heparin salts having an average molecular weight of less than about 8000 Daltons (Da). The fluid sample may be obtained from the blood, including a fluid fraction thereof, of an individual, such as an individual that is a mammal, including a human. The individual may be a patient undergoing a procedure or therapy where coagulation is a concern. Those individuals may be receiving periodic administration or heparin. In an aspect, the fluid sample may be native whole blood; citrated whole blood, citrated plasma, or citrated platelet rich plasma.

The terms “first parameter” and “second parameter” are used broadly herein to refer to quantification of the Coagulation Reaction Phase and Clot Formation Phase. Accordingly, in an aspect the first parameter characterizes Coagulation Reaction Phase and the second parameter characterizes Clot Formation Phase. “Characterization” refers to the parameters that change with changing heparin concentration. Depending on the instrument used, the specific parameter variable changes, although the general nature of the parameter matched to a phase remains, in principle, the same. For this reason, the parameter measurement is by any instrument known in the art that provides information related to at least one phase of the two-phase clotting model illustrated by FIG. 1.

An example of a suitable first fluid parameter includes a measurement that characterizes the Coagulation Reaction Phase, and may be expressed in terms of a time, including reaction time or defined fraction thereof. This measurement is typically a time. An example of a suitable second fluid parameter includes a measurement that characterizes a Clot Formation Phase. The second fluid parameter is typically expressed in terms of a time or a rate.

Any one of number of instrumentation may be used to calculate or measure the first or second parameters that is a Clot Reaction Phase parameter or Clot Formation Phase parameter, respectively, by generating a physical parameter that is measured by a viscosity measurement; elastic measurement; optical transmission measurement; optical diffusion measurement; or ultrasonic measurement.

Any one of a number of devices may be used to calculate a parameter that characterizes the Coagulation Reaction Phase including a generic clot timer, automated optical coagulation analyzer, TEG, Rotem, Sonoclot Analyzer, Thromboscope, or ultrasonic coagulation analyzer. Accordingly, the specific first parameter depends on the instrument used to measure the first parameter. In particular, the devices use many different terms to quantify the Coagulation Reaction Phase including: prothrombin time, International Normalized Ratio, partial thromboplastin time, activated partial thromboplastin time, activated clotting time, Thromboelastography R, Thromboelastography R+k, Sonoclot ACT, Sonoclot Onset Time, Rotem (RT, CT, CFT), Thromboscope Lag time, or an optical property obtained from an optical transmission or chromogenic plasma coagulation analyzer.

Many devices may be used to calculate the Clot Formation Phase parameter including: automated optical coagulation analyzer TEG, Rotem, Sonoclot Analyzer, Thromboscope, ultrasonic coagulation analyzer. These devices use many different terms to quantify the Clot Formation Phase including: Thromboelastography (k; α; MA; T; A30 or A60); Sonoclot Clot Rate; Rotem (MCF; MCF-t; CFT; α; A5, A10); Thromboscope (Time to Peak; Time to Peak—Lag Time; Peak; ETP; slope of calibrated automated thrombogram; maximum acceleration of calibrated automated thrombogram); or a parameter derived from a clot curve developed from an optical transmission or chromogenic plasma coagulation analyzer.

From the first and second parameters, the respective intermediate results may be heparin concentration estimates, as disclosed herein from each of the parameters. The intermediate result and its corresponding variance estimate may be calculated from a dataset of parameter results collected from blood samples from multiple patients or healthy individuals wherein the heparin concentration is known. The heparin concentration may be known either by adding a predetermined amount of heparin to a controlled volume of whole blood or measuring the heparin concentration of the plasma with more expensive laboratory reference tests such as an anti-Xa assay. Both the intermediate result and variance estimate are functions of the individual parameter.

For a heparin concentration intermediate result and corresponding variance estimate a final heparin concentration determination is calculated as a weighted average of the individual heparin concentration estimates. The weight assigned to an individual heparin concentration estimate is the inverse of the variance of the heparin concentration estimate. This variance weighted average offers two significant benefits. First, the variance of the weighted average will be less than the variance of the individual estimates for most datasets. Second, more weight is placed on the estimate that has less variance. Some heparin concentration estimates have less variance at lower levels of heparin concentration and other heparin concentration estimates have less variance at higher heparin concentrations. This weighted average approach compensates for the changes in heparin concentration estimate variance across the range of heparin concentrations.

Heparin concentration determination may be reported in International Units (IU) of heparin per mL whole blood or IU of heparin per mL of plasma.

“Heparin response imbalance” refers to an individual that does not respond to heparin in an expected manner, suggesting a defect within the coagulation cascade related to the heparin-mediated pathway. A heparin response imbalance may be assessed or calculated from multiple heparin concentration estimates. The response imbalance may be quantified in different ways including: calculating the difference between two individual heparin concentration estimates; calculating a normalized difference between two individual heparin concentration estimates; or calculating a ratio of two individual heparin concentration estimates.

A heparin response imbalance may be used to identify samples that respond abnormally to heparin. A lower than expected heparin concentration estimate based on a Coagulation Reaction Phase parameter in comparison to a heparin concentration estimate based on a Clot Formation Rate parameter would indicate a potential deficiency in factors contributing to the Coagulation Reaction Phase. A higher than expected heparin concentration estimate based on the Coagulation Reaction Phase parameter in comparison to a heparin estimate based on a Clot Formation Phase parameter would indicate a potential deficiency in factors contributing to the Clot Formation Phase. In this manner, the measurement of the two parameters and related intermediate results that are heparin concentration estimates provides a platform for assessing whether or not an individual has a heparin response imbalance.

In another embodiment, any of the methods disclosed herein may be practiced on two samples from an individual, such as by dividing an individual sample into two samples, a first and second sample, wherein one of the samples may contain heparin and the other sample contains no active heparin. No active heparin refers to a fluid sample in which no heparin has been added. Alternatively, no active heparin refers to at sample in which at least part or at least substantially all of the added heparin has been either removed or inactivated such as by a filter or by a chemical inactivation.

In another embodiment, the invention is a system for carrying out any of the methods provided herein to determine a heparin parameter in a fluid sample.

### BRIEF DESCRIPTION OF THE DRAWINGS

FIG. 1: Two Phase Clotting Model Illustrating Coagulation Reaction Phase and Clot Formation Phase.

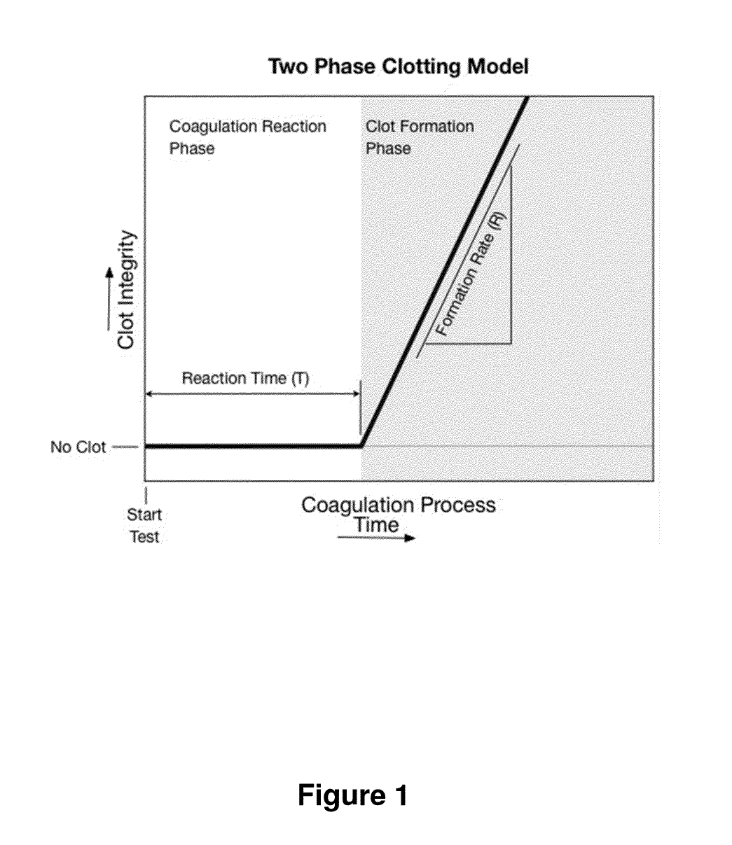

FIG. 2: Heparin Effect on a Sonoclot™ Analyzer.

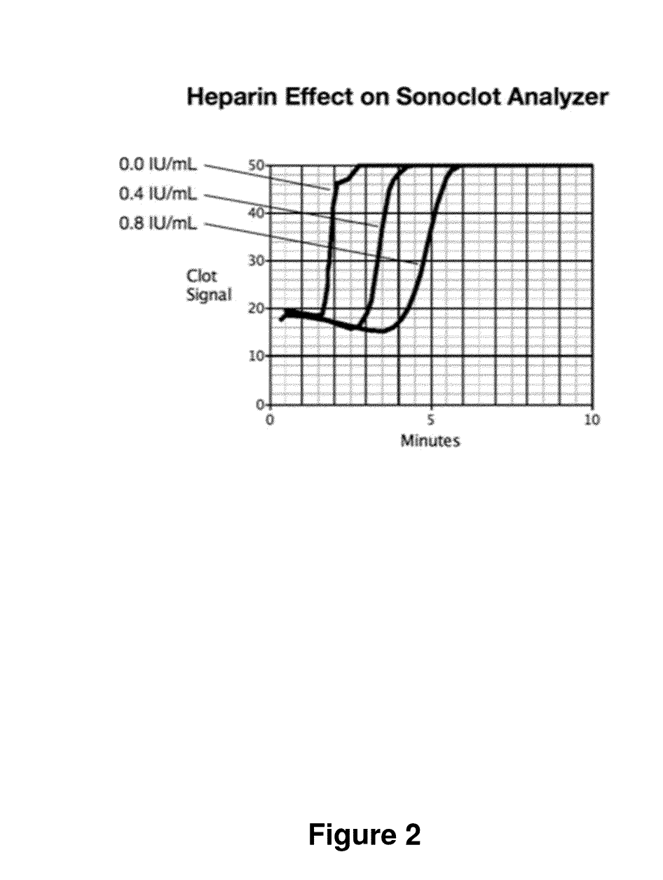

FIG. 3: Heparin Effect on Coagulation Reaction Time.

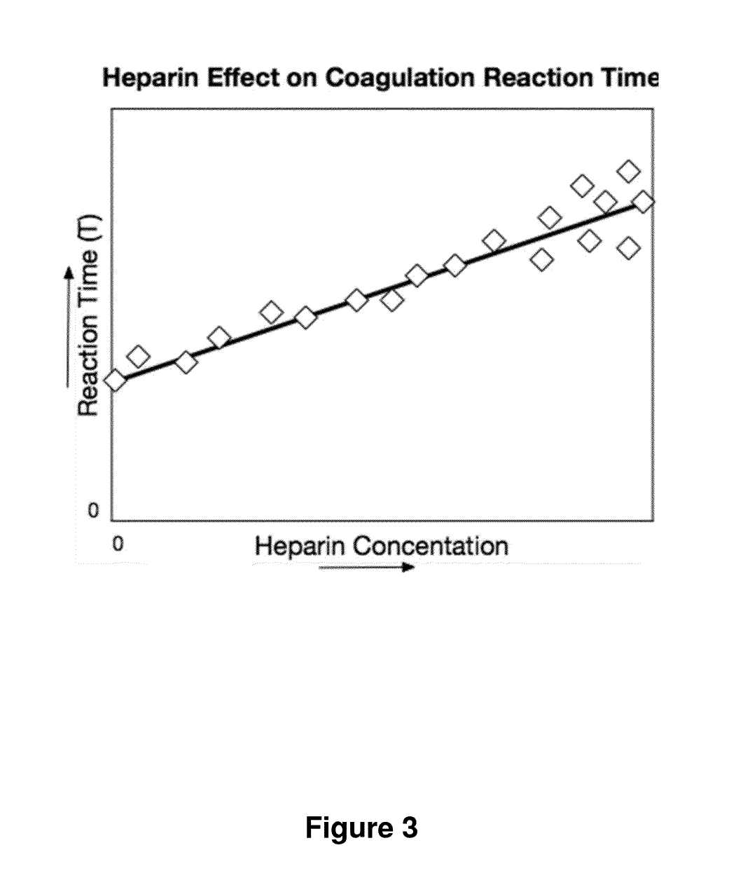

FIG. 4: Heparin Effect on Clot Formation Rate.

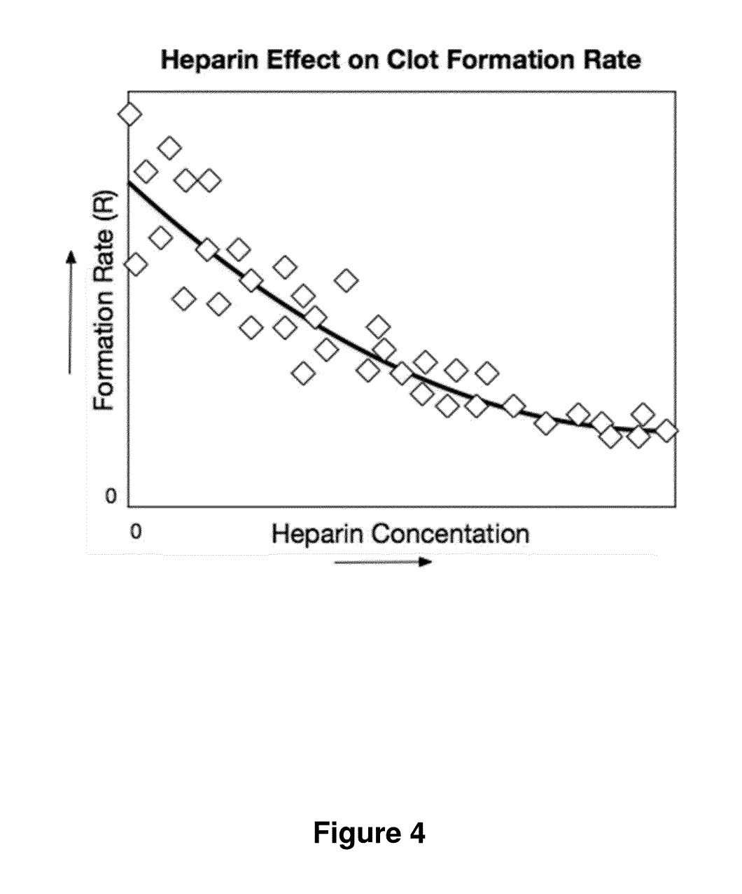

FIG. 5: Heparin Concentration Determination Method—Single Channel Whole Blood Embodiment Flow Chart.

FIG. 6: Heparin Concentration Determination Method Single Channel Plasma Embodiment Flow Chart.

FIG. 7: Single Channel Embodiment Chi Square Results—Whole Blood.

FIG. 8: Single Channel Embodiment Chi Square Results—Plasma.

FIG. 9: Heparin Concentration Determination Method Two Channel Whole Blood. Embodiment Flow Chart

FIG. 10: Heparin Concentration Determination Method Two Channel Plasma Embodiment Flow Chart.

FIG. 11: Two Channel Chi Square Results—Heparin and Heparin Neutralized Whole Blood.

FIG. 12: Two Channel Chi Square Results—Heparin and Heparin Neutralized Plasma.

FIG. 13: Heparin Concentration Calibration Equation Flow Chart—Whole Blood.

FIG. 14: Heparin Concentration Calibration Equation Flow Chart—Plasma.

FIG. 15: Statistical Analysis to Determine the Heparin Calibration Equation, H_{W}(T), and the Associated Standard Error, σH_{W}(T)—Whole Blood.

FIG. 16: Statistical Analysis to Determine the Heparin Calibration Equation, H_{W}(T), and the Associated Standard Error, σH_{P}(T)—Plasma.

FIG. 17: Statistical Analysis to Determine the Heparin Calibration Equation, H_{P}(R), and the Associated Standard Error, σH_{P}(R)—Plasma.

FIG. 18: Heparin Concentration Method Precision Performance Summary.

### DETAILED DESCRIPTION OF THE INVENTION

Explicit definition of various terms and variables are summarized in the Table of Terms, including Tables 1-5.

### Example 1

### Single Channel Embodiment

A simple embodiment of the heparin concentration or heparin response imbalance method is implemented on a single channel instrument. The implementation requires an instrument capable of measuring multiple aspects of the two-phase coagulation model, such as T and R results. The instrument calculates a heparin concentration from the T and R results. The instrument can be calibrated to calculate heparin concentration estimates in either whole blood or plasma. The process utilizes two separate procedures: a calibration procedure that derives the relationships between T and R results and heparin concentration in either whole blood or plasma, and a test analysis procedure that runs a test, calculates T and R, and then calculates a heparin concentration estimate based on the T and R results.

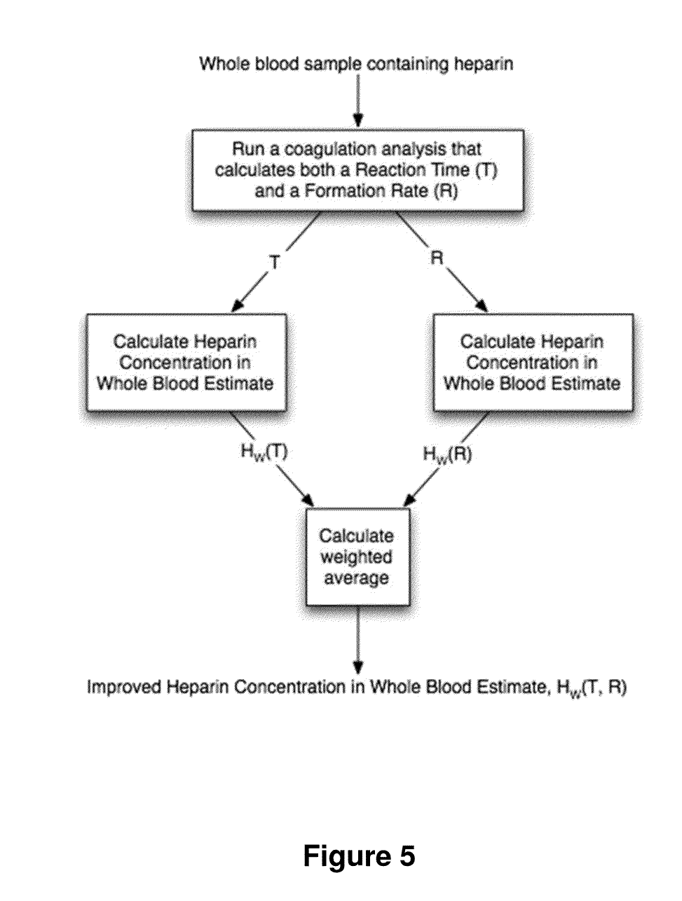

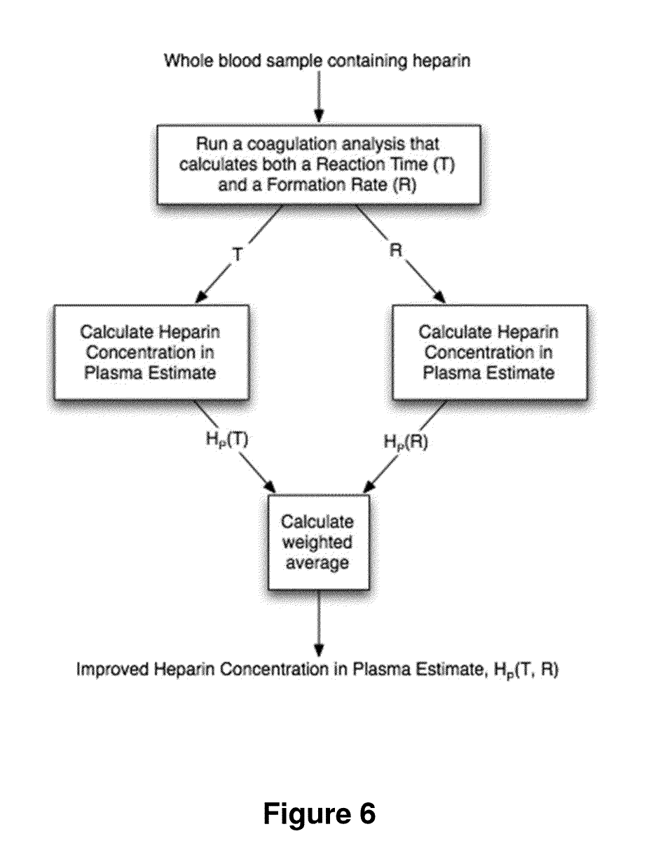

The single channel embodiment procedure for running a test and calculating a heparin concentration in whole blood is described in the flow chart contained in FIG. 5. A patient whole blood sample containing heparin is run on an analyzer capable of calculating both T and R. A heparin estimate equation within the instrument, H_{W}(T), calculates a heparin concentration estimate based on the T result. Another heparin concentration estimate equation within the instrument, H_{W}(R), calculates a second heparin concentration estimate based on the R result. As used herein, T and R are examples of a “first parameter” and a “second parameter” that vary with heparin concentration and heparin concentration estimates. H_{W}(T) and H_{W}(R) are examples of a “first intermediate result” and a “second intermediate result.” These two heparin concentration estimates are used to calculate a weighted average heparin concentration estimate, H_{W}(T,R), which is an example of a heparin parameter. The weights for H_{W}(T) and H_{W}(R) used in calculating the weighted average are based on statistical variance assessed during calibration and discussed later in this description. With proper factory calibration of the weighting functions, the combined heparin estimate, H_{W}(T,R), achieves higher measurement precision than either H_{W}(T) or H_{W}(R) estimates alone. FIG. 6 shows an equivalent process to calculate a heparin concentration in plasma rather than whole blood. Note, a whole blood test sample is used; the heparin concentration estimate equations, H_{P}(T), H_{P}(R), and the resulting variance weighted average, H_{P}(T,R), perform the conversion into heparin concentration in plasma rather than whole blood.

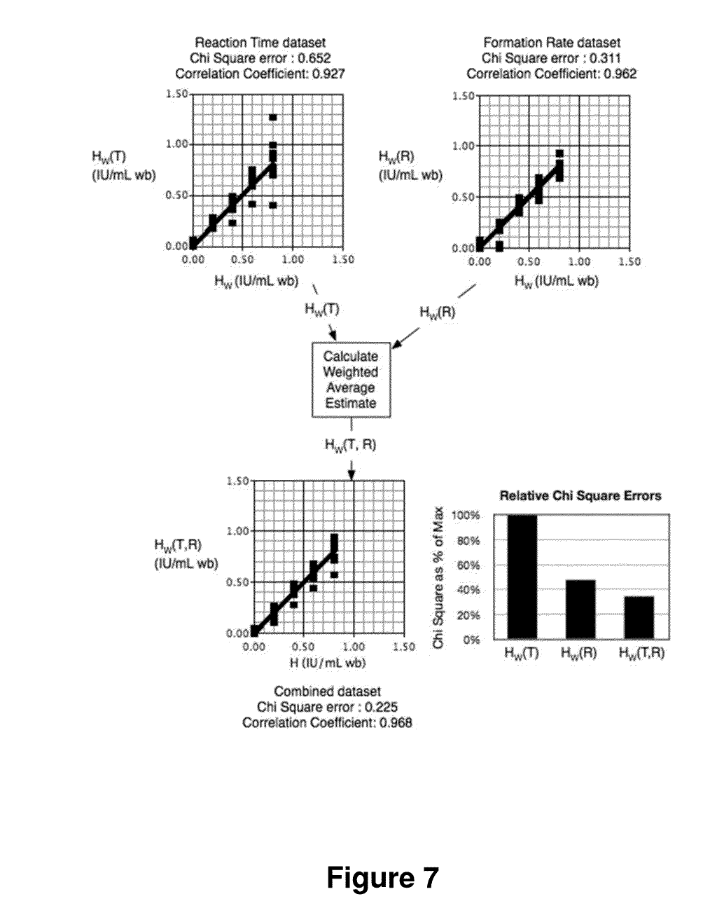

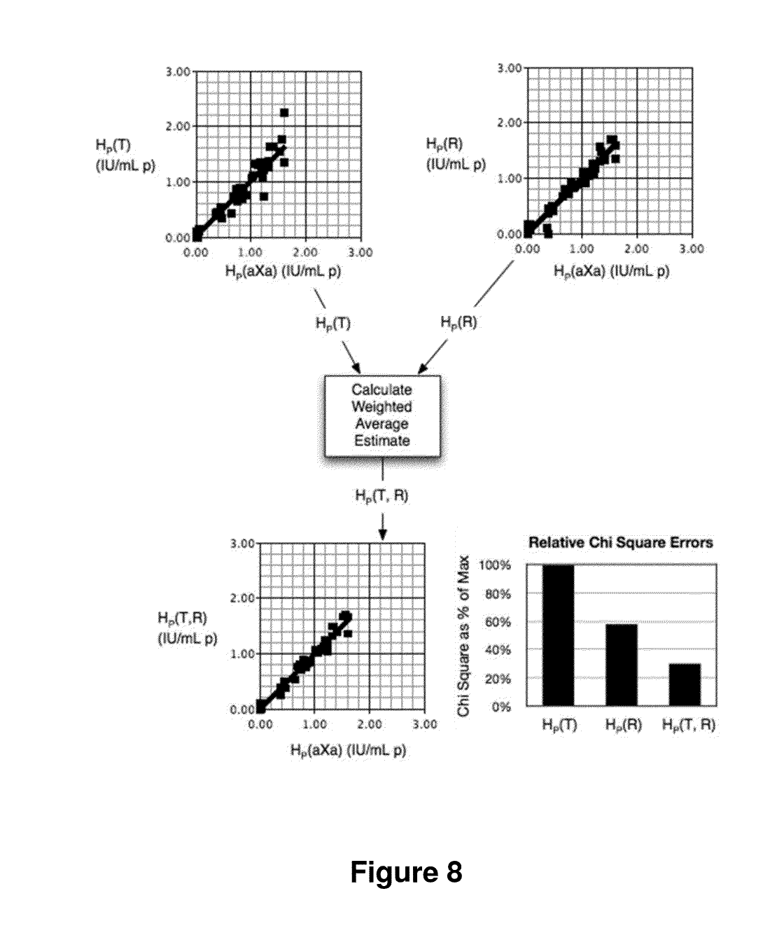

The improvements in precision are illustrated on an actual data test population shown in the graphs in FIGS. 7 and 8. For this dataset, results are obtained using a Sonoclot Analyzer; the ACT result is used as T; the Clot Rate result is used as R.

FIG. 7 shows results for heparin concentration determination in whole blood. In each of the three graphs, the X axis is the known amount of heparin and the Y axis is the estimated heparin concentration, H_{W}(T), H_{W}(R), or H_{W}(T,R) based on T, R, or both T and R respectively. The amount of heparin is known because the blood sample did not contain any heparin when it was drawn from the donor and a known amount of heparin is added to each sample prior to testing.

For a perfect estimation without any error, all data points fall on the identity line. The error in the heparin concentration measurement for each data point is the distance along the Y axis between a data point and the identity line. One measure of the quality of a prediction is the sum of the squared error for each datapoint. This statistic is referred to as the Chi Square. The lower the Chi Square statistic for a dataset, the more precise the measurement. For this dataset, the H_{W}(T) Chi Square is 0.652; the H_{W}(R) Chi Square is 0.311; and the combined heparin estimate, H_{W}(T,R), has a Chi Square of 0.225. H_{W}(T,R) achieves a higher precision for measuring heparin concentration than either of the individual test results alone. Overall, the heparin concentration method achieves an improvement over H_{W}(T) with a 65% reduction in the Chi Square for this initial sample population (see FIG. 7 bottom right panel). Careful examination of the data shows that the variance weighted average within the H_{W}(T,R) calculation put more emphasis on the H_{W}(T) at low levels of heparin and more emphasis on the H_{W}(R) at higher levels of heparin. The Two Phase Coagulation Model implementation achieves dramatic improvement in precision over the heparin concentration estimates based on conventional clot timer results alone.

FIG. 8 shows a similar collection of results as FIG. 7 except the known amount on the X axis has been changed from heparin concentration in whole blood to heparin concentration in plasma as measured by an anti-Xa laboratory test. Again, the combined prediction of H_{P}(T,R) is better than either of the individual predictions, H_{P}(T) or H_{P}(R).

It is important to note that an instrument can be calibrated to report heparin concentrations in either whole blood, plasma, or both. Some clinical users may prefer to monitor whole blood heparin concentration while laboratory users may prefer to monitor plasma heparin concentration.

Testing on the instrument can be done using native whole blood, citrated whole blood, or citrated plasma as long as the proper calibration equations are used for the type of blood sample being tested.

Heparin response imbalance can be determined from calculating a numerical comparison of either H_{P}(T) and H_{P}(R) or H_{W}(T) and H_{W}(R). One quantitative approach is a normalized difference:

HRI_{P}(T,R)=H_{P}(T)−H_{P}(R))/(H_{P}(T)+H_{P}(R))

Higher values of this version of HRI_{P}(T,R) indicate a deficiency in the Clot Formation Phase. Lower values indicate a deficiency in the Coagulation Reaction Phase. These results should only be reported for heparin concentrations above a minimum value to avoid numerical instability by using too small a divisor.

Alternately, the comparison is a ratio:

HRI_{P}(T,R)=H_{P}(T)/(H_{P}(R)+σH_{P}(T)+σH_{P}(R))

With this ratio, numerical instability is avoided by adding the associated standard errors to the denominator. Higher results indicate a deficiency in the Clot Formation Phase. A similar heparin response imbalance specific to deficiencies in the Coagulation Reaction Phase is:

HRI_{P}(T,R)=H_{P}(R)/(H_{P}(T)+σH_{P}(T)+σH_{P}(R))

### Example 2

### Two Channel Embodiment

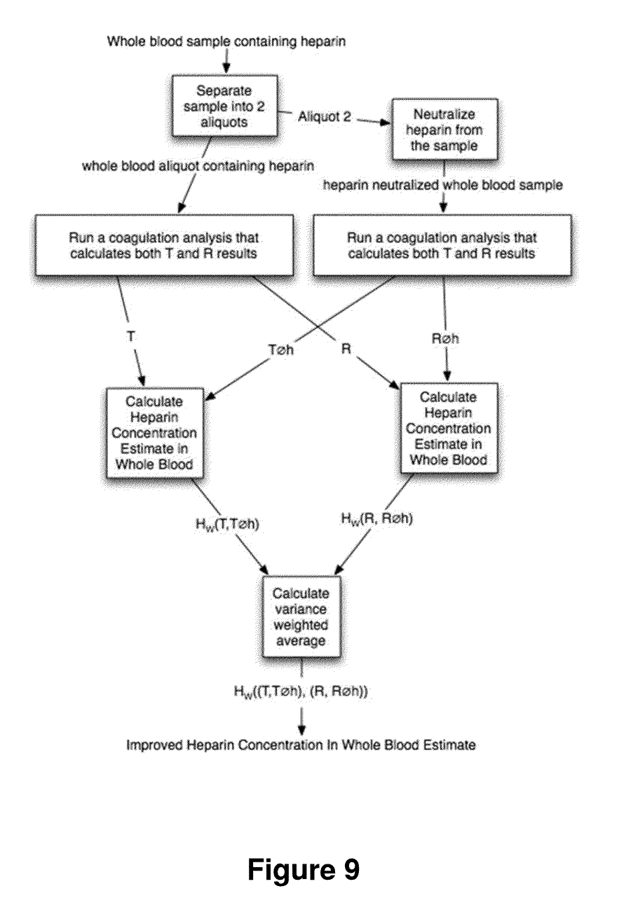

FIG. 9 shows another embodiment of the method. Here, two tests are run for each blood sample: one sample run on the instrument and a second sample run on the instrument after first neutralizing or removing the heparin. In this test analysis, the two Reaction Times, T and TØh, the Reaction Time without heparin, are used to make an improved estimate, H_{W}(T,TØh), the heparin concentration estimate that characterizes the Coagulation Reaction Phase performance. T and TØh results are combined to generate an estimate that corrects for patient to patient variability in T when no heparin is present. In this embodiment, H_{W}(T,TØh) is implemented using ΔT, the difference: T−TØh; thus H_{W}(T,TØh) is implemented with H_{W}(ΔT). H_{W}(T,TØh) has lower variance than H_{W}(T) at low heparin levels. However, H_{W}(T,TØh) has less variance than H_{W}(T) only at low heparin concentrations.

The two Reaction Rates, R and RØh, the Formation Rate without heparin, are used to make an improved estimate of heparin concentration based on the Clot Formation Phase of the Two Phase Coagulation Model. The R and RØh results are combined to generate an estimate that corrects for patient to patient variability in the R when no heparin is present. While T and TØh are combined with a simple linear relationship, R is non-linear and requires a more complex numerical model to achieve useful precision. The variance of R is greatest without heparin and decreases with increasing heparin concentration. The variance at low heparin levels can be substantially reduced by using a variable, Rate Ratio (RR), the ratio RØh/R. This variable has small variance at low heparin levels and larger variance at higher heparin levels. An improved characterization of H_{W}(R,RØh) is obtained across both low and higher heparin concentration levels by using a variance weighted average of the two separate heparin concentration estimates, H_{W}(R) and H_{W}(RR) to create a combined heparin estimate equation H_{W}(R,RØh).

In FIG. 9, the two heparin concentrations estimates, H_{W}(T,TØh), and H_{W}(R,RØh) are used to create a combined heparin concentration estimate, H_{W}((T,TØh), (R,RØh)) using a variance weighted average.

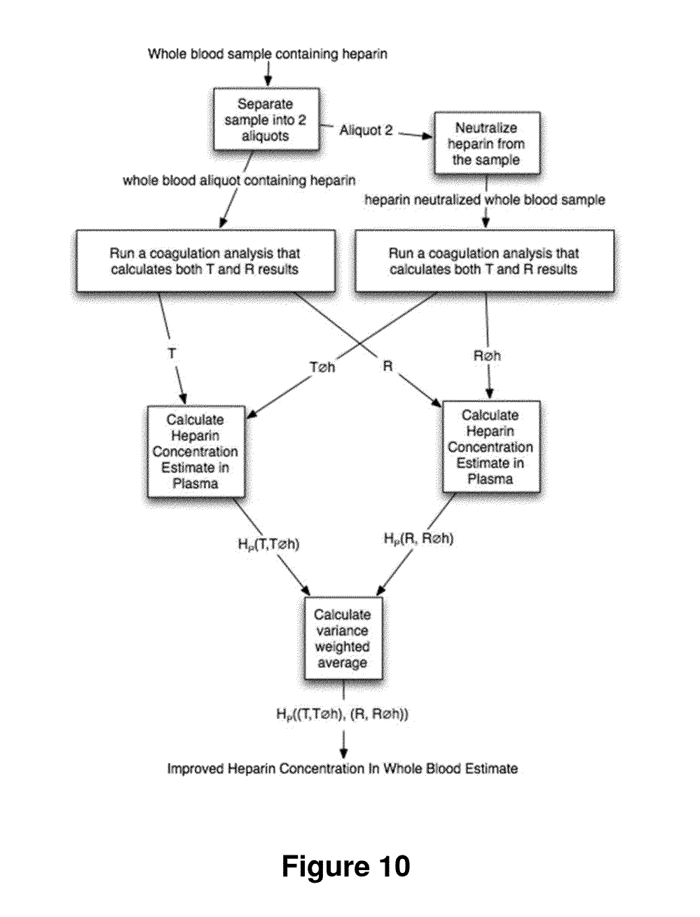

The instrument incorporates the heparin concentration equations, H_{W}(T) and H_{W}(R), which are the same equations used in the single test embodiment. Two additional heparin calibration equations, H_{W}(T,TØh) and H_{W}(R,RØh) are included in this embodiment. A final heparin concentration estimate, H_{W}((T,TØh), (R,RØh)), is obtained using these two heparin concentration estimates to calculate a variance weighted average of the individual estimates. FIG. 10 shows an equivalent process to calculate a heparin concentration in plasma rather than whole blood.

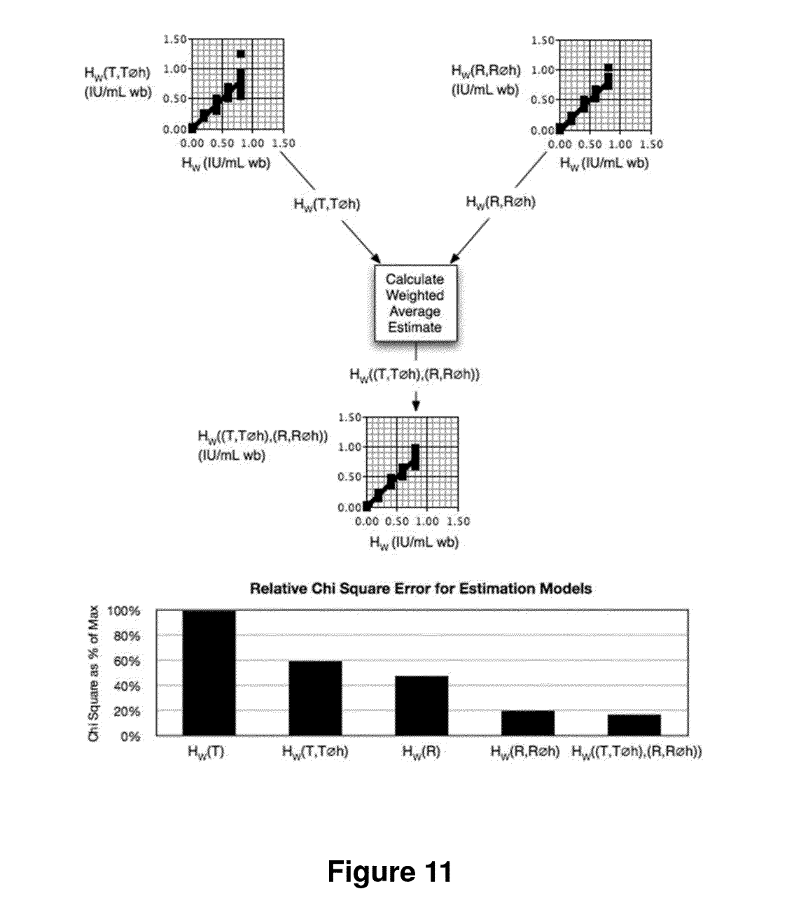

FIG. 11 shows the improvement in precision obtained on a dataset using test results on heparin and heparin neutralized blood samples to estimate heparin concentrations in whole blood. In this embodiment H_{W}(T,TØh) is implemented using H_{W}(ΔT), and H_{W}(R,RØh) is implemented with a variance weighted average of the two separate heparin concentration estimates, H_{W}(R) and H_{W}(RR). The combined heparin concentration estimate reduced the Chi Square of the basic Clot Timer result by over 80% for this dataset. It is noted that ΔT testing is available in commercial products; H_{W}(ΔT) achieved a 40% Chi Square reduction which is significantly less than the 82% Chi Square reduction achieved herein with H_{W}((T,TØh), (R,RØh)).

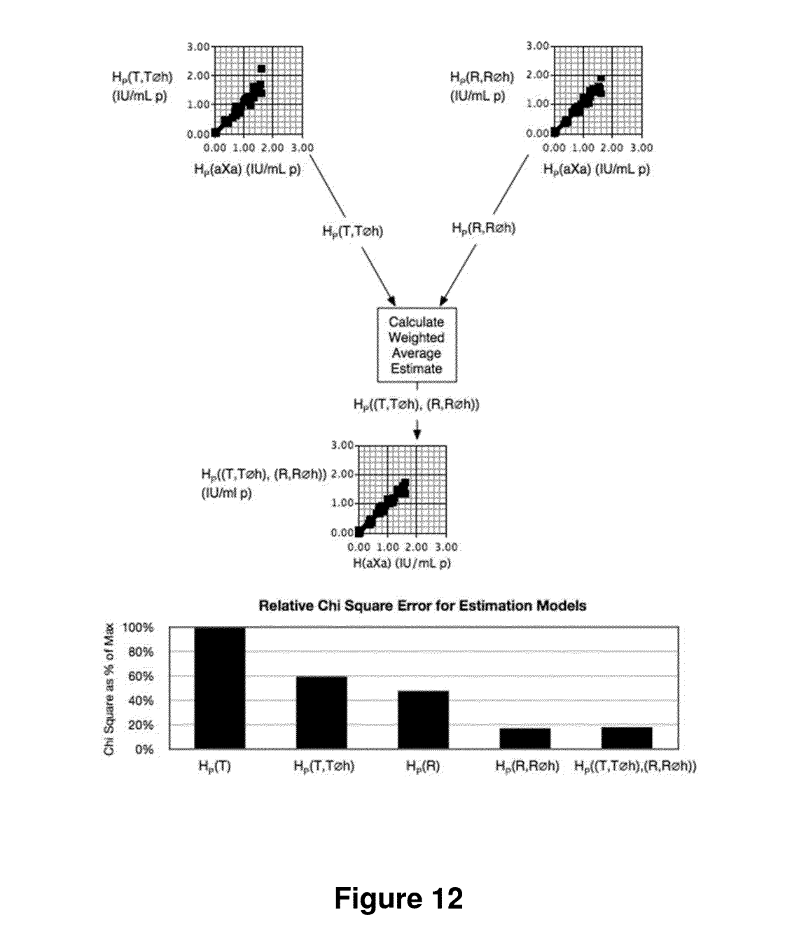

FIG. 12 shows the improvement in precision obtained on a dataset using the sample data as in FIG. 11 except the X axis is changed from heparin concentration in whole blood to heparin concentration is plasma as measured by an anti-Xa laboratory test. The combined estimate reduces the Chi Square of the basic Clot Timer result by 77% for this dataset, whereas prior art performance as quantified with H_{W}(ΔT), achieved only a 33% Chi Square improvement.

Heparin response imbalance can be determined from calculating a numerical comparison of (H_{P}(T,TØh) and H_{P}(R,RØh)) or (H_{P}(T,TØh) and H_{P}(R,RØh)) similarly to the single channel embodiment.

### Example 3

### Calibration Equations

FIGS. 7, 8, 11, and 12 show examples where an improved estimation of heparin concentration is obtained from two or more estimates of the heparin concentration. In these examples heparin concentration estimation equations are used to determine two or more heparin concentration estimates from [T, R] or [T, R, TØh, RØh] test results. Additionally, a variance weighted average of the individual heparin concentration estimates is used to determine a final heparin concentration result. The most precise combined estimate from multiple independent estimates is achieved when the weight used for an individual estimate is equal to the inverse of the variance of the estimate. Estimates with lower variance contribute greater weight than estimates with higher variance. During calibration, the standard error of each intermediate result is estimated as a function of the corresponding initial heparin parameter. The weight is the inverse of the square of the standard error.

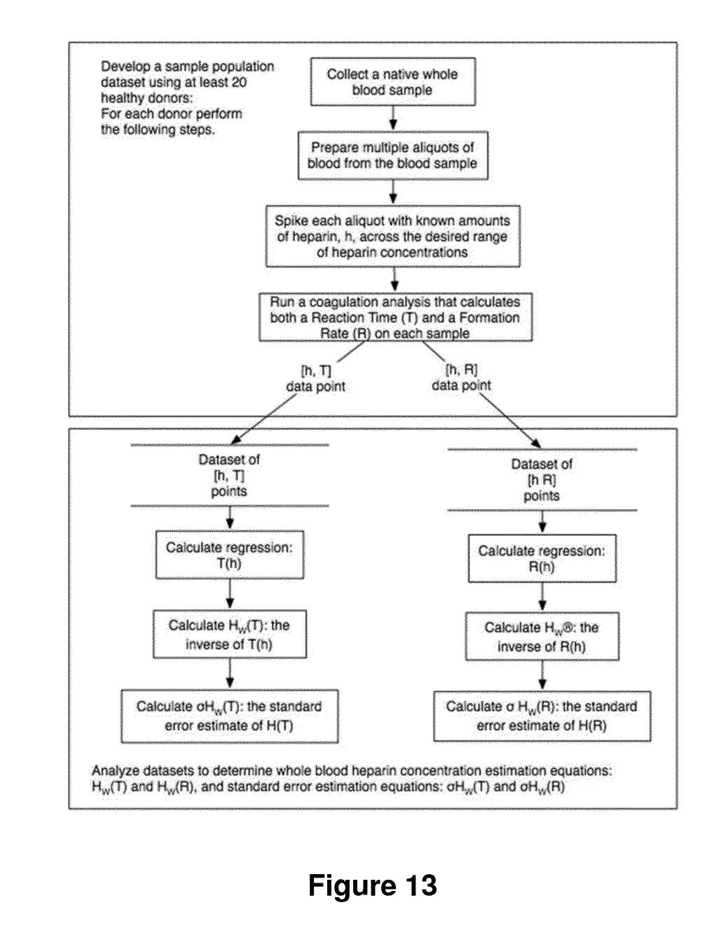

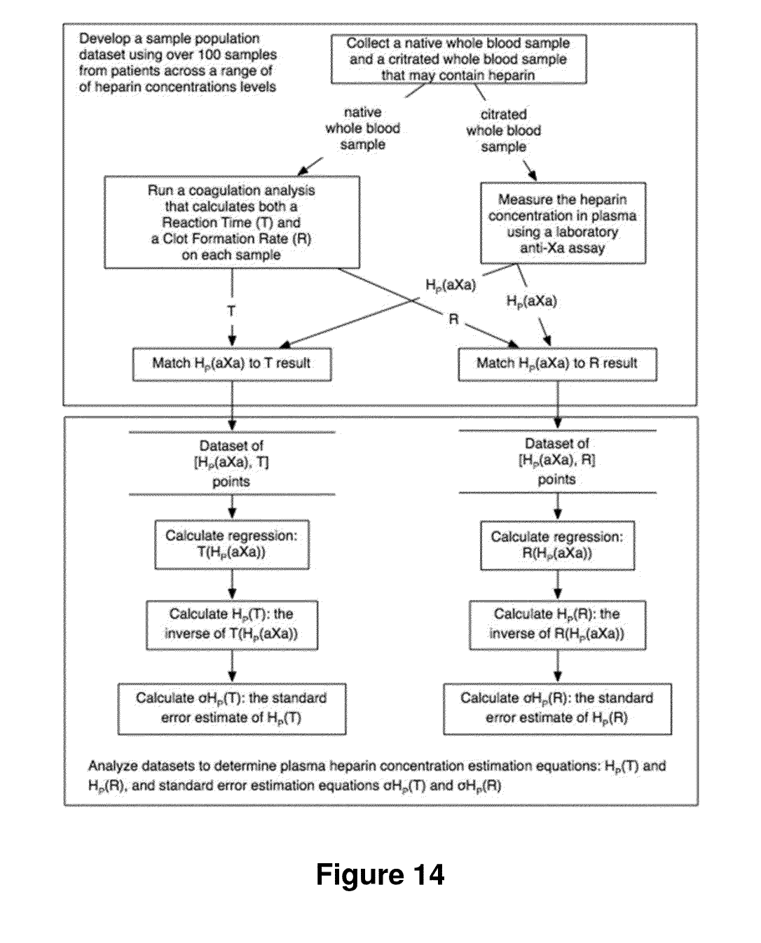

The heparin concentration estimate equations and standard error estimates are calculated using regression analysis, statistics, and algebra. This process involves two steps. First, a dataset of points that contain both T and R results and known amounts of heparin concentrations is collected. Next, the resulting dataset is numerically analyzed to calculate heparin estimates and standard errors of those estimates. The flowchart in FIG. 13 details the process for collecting and analyzing a dataset for T and R results and developing calibration equations and standard errors for H_{W}(T) and H_{W}(R) to determine heparin concentrations in whole blood. FIG. 14 details the process for collecting and analyzing a dataset for T and R results for H_{P}(T) and H_{P}(R) to determine heparin concentrations in plasma.

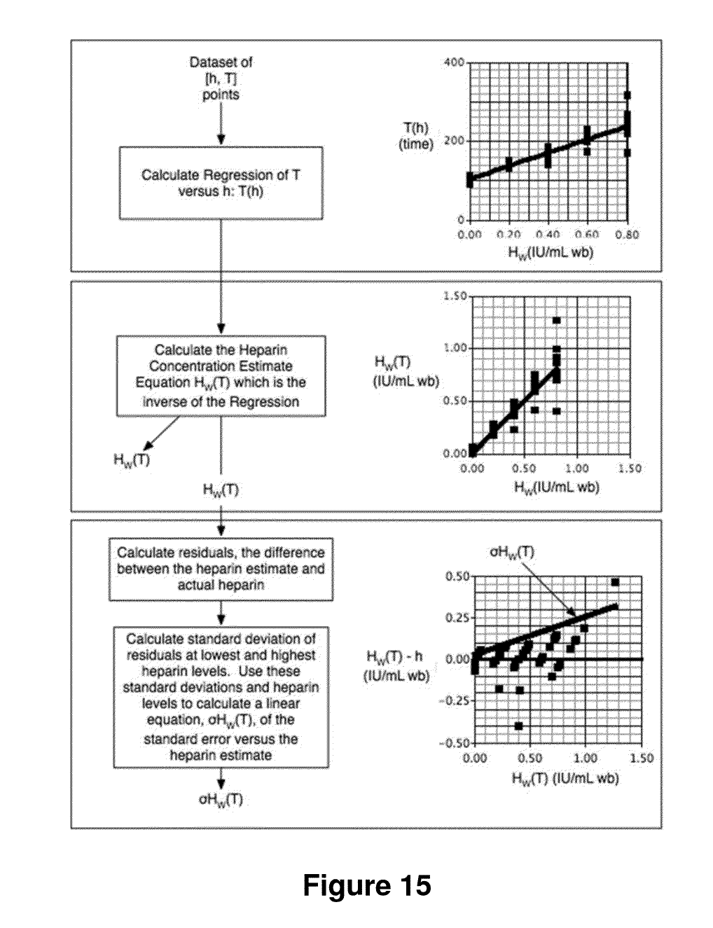

The statistical analysis to determine the heparin calibration equation, H_{W}(T), and the associated standard error, σH_{W}(T), for heparin concentrations in whole blood is illustrated in FIG. 15. A linear regression is used to calculate the estimate T(h). The resulting calibration equation, H_{W}(T), is the inverse of T(h). The standard error equation, σH_{W}(T), is estimated as a straight line using the standard deviation of the estimate error at two points, heparin=0 and heparin=maximum concentration used for the calibration.

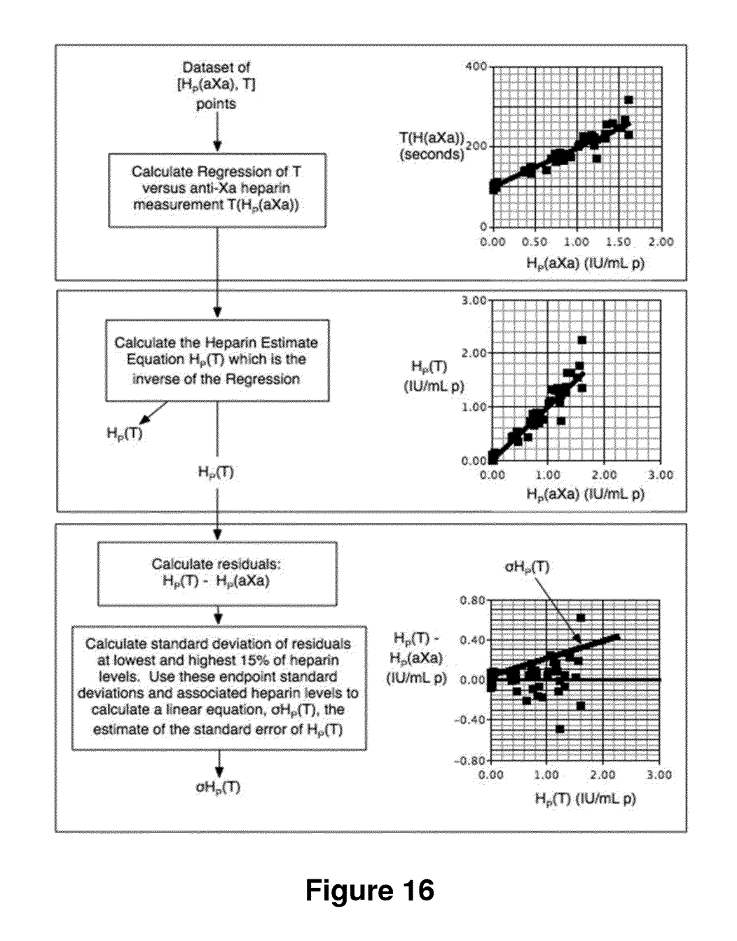

FIG. 16 shows the statistical analysis to determine the heparin calibration equation, H_{P}(T), and the associated standard error, σH_{P}(T), for heparin concentration estimates in plasma. The only difference between heparin concentration calibration in plasma rather than whole blood is that the X axis contains heparin levels in plasma as measured by anti-Xa analysis. The X data is not stacked as fixed concentrations but spread out across the X axis.

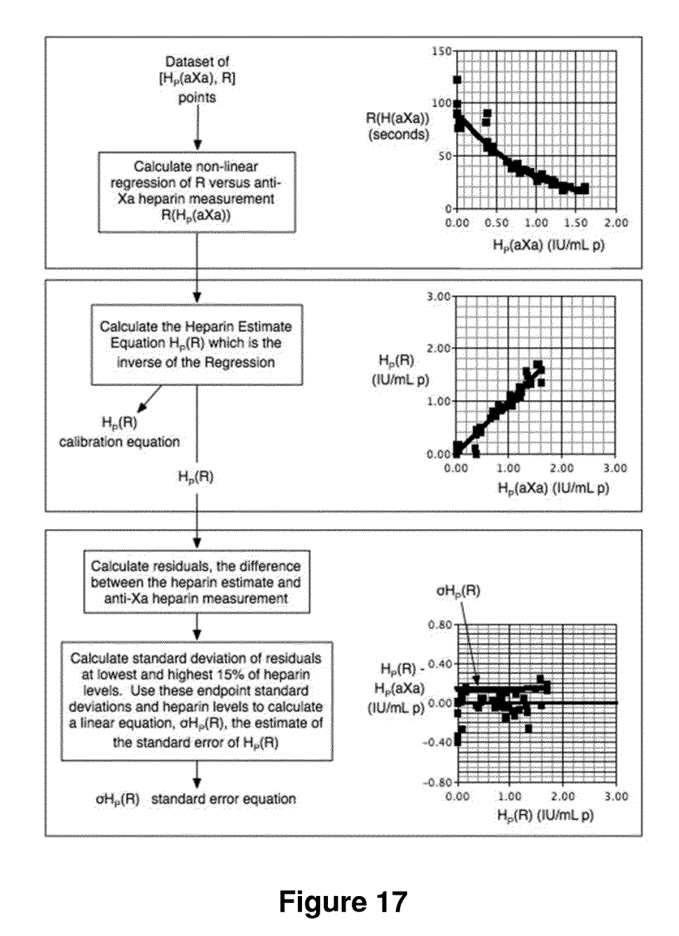

FIG. 17 shows the statistical analysis to determine the heparin calibration equation, H_{P}(R), and the associated standard error, σH_{P}(R), for heparin concentration estimates in plasma. The major difference between the statistical analysis of T and R is that T is effectively modeled as a linear relationship whereas R is modeled as a second order polynomial. Calculating the inverse of a second order model requires solving a quadratic equation and using only the result that falls within the range of expected heparin concentrations. FIG. 17 shows results for plasma; an equivalent analysis for whole blood achieves similar results.

For calibrating instruments that incorporate heparin neutralized results, the only required additional steps are to add heparin neutralized data, TØh and RØh, to the dataset and expanding the statistical analysis to include either H_{W}(T,TØh) and H_{W}(R,RØh), or H_{P}(T,TØh) and H_{P}(R,RØh) results. The process of first calculating a regression, then inverting the regression generates the heparin estimate equations. The standard error estimates for these heparin estimate equations is also calculated following the same numerical analysis as used for T and R results.

### Example 4

### Two Channel—Whole Blood Heparin Concentration

A Sonoclot Analyzer can be used to implement either a single channel or a two channel embodiment of the heparin concentration determination method. The two channel embodiment is an extension of the single channel embodiment. The embodiment of the two channel heparin concentration method based on a Sonoclot Analyzer is described in the following step by step procedures. The single channel embodiment is not presented since the two channel embodiment covers the single channel embodiment and adds heparin neutralized test results to the analysis.

Two separate procedures are required: a first procedure to calibrate an instrument for calculating a heparin concentration, and a second procedure to measure the heparin concentration of a sample containing an unknown concentration of heparin. The Sonoclot ACT result is used as the T and the Sonoclot CR result is used as the R.

In this embodiment, H_{W}(T,TØh) is implemented with H_{W}(ΔT). H_{W}(R,RØh) is implemented with a variance weighted average of H_{W}(R) and H_{W}(RR).

Heparin Concentration Calibration for Test Reagent
1. Collect a native whole blood sample from a healthy donor.
2. Immediately spike the whole blood samples with known amounts of heparin
3. Run the spiked heparin sample on a Sonoclot Analyzer following manufacturer's instructions on two Sonoclot Analyzer tests: a P2-1 and a P2-2. The Sonoclot ACT run on the P2-1 is T. The Sonoclot ACT run on the P2-2 is TØh. The Sonoclot CR run on the P2-1 is R; the Sonoclot CR run on the P2-2 is RØh.
4. Repeat this data collection process on at least 30 donors:
5. Calculate the linear and 2nd order regressions
   R(h) (2nd order)
   ΔT(h) (linear)
   RR(h) (2nd order)
6. Calculate the regression inverses—these are calibration equations
   H_{W}(R)
   H_{W}(ΔT)
   H_{W}(RR)
7. Calculate standard deviations of the known heparin concentration minus the heparin estimates, H_{W}(T), H_{W}(R), and H_{W}(RR) at 0 and maximum heparin concentrations:
   σH_{R@H=0}, standard deviation for the [h,H(R)] points for h=0
   σH_{R@H=MAX}, standard deviation for the [h,H(R)] points for h=maxi mum heparin concentration
   σH_{ΔT@H=0}, standard deviation for the [h,H(ΔT)] points for h=0
   σH_{ΔT@H=MAX}, standard deviation for the [h,H(ΔT)] points for h=maxi mum heparin concentration
   σ_{RR@H=0}, standard deviation for the [h,H(RR)] points for h=0
   σH_{RR@H=MAX}, standard deviation for the [h,H(RR)] points for h=maxi mum heparin concentration
8. Calculate standard error calibration equations from standard deviation points
   σH_{W}(R), a linear equation of the standard error across the range of heparin concentrations based on σH_{R@H=0} and σH_{R@H=MAX}
   σH_{W}(ΔT), a linear equation of the standard error across the range of heparin concentrations based on σH_{ΔT@H=0} and σ_{ΔT@H=MAX}
   σH_{W}(RR), a linear equation of the standard error across the range of heparin concentrations based on σH_{RR@H=0} and σ_{RR@H=MAX}

### Heparin Concentration Measurement

9. Collect native whole blood sample that may contain heparin
10. Run a Sonoclot Analysis using both a P2-1 and P2-2 activated test.
11. The Sonoclot Analyzer calculates three heparin concentration estimates
   H_{W}(R)
   H_{W}(ΔT)
   H_{W}(RR)
12. The Sonoclot Analyzer calculates variances for each estimate:
   σH_{W}²(R)
   σH_{W}²(ΔT)
   σH_{W}²(RR)
   These variances are the square of the standard error estimates.
13. The Sonoclot Analyzer combines H_{W}(R) and H_{W}(RR) into a single estimate, H_{W}(R,RR) by first calculating a combined weight for each estimate using standard algebra for calculating a weighted average with each individual weight the inverse of the variance:
   w_{R}=1/σH_{W}²(R)/(1/σH_{W}²(R)+1/σH_{W}²(RR))
   w_{RR}=1/σH_{W}²(RR)/(1/σH_{W}²(R)+1/σH_{W}²(RR))
14. The Sonoclot Analyzer calculates H_{W}(R,RR):
   H_{W}(R,RR)=w_{R}H_{W}(R)+w_{RR}H_{W}(RR)
15. The Sonoclot Analyzer calculates σH_{W}²(R,RR):
   σH_{W}²(R,RR)=w_{R}σH_{W}²(R)+w_{RR}σH_{W}²(RR)
16. The Sonoclot Analyzer calculates weights for final heparin concentration measurement:
   w_{ΔT}=1/σH_{W}²(ΔT)/(1/σH_{W}²(ΔT)+1/σH_{W}²(R,RR))
   w_{R,RR}=1/σH_{W}²(R,RR)/(1/σH_{W}²(ΔT)+1/σH_{W}²(R,RR))
17. The Sonoclot Analyzer calculates the final heparin concentration measurement as a variance weighted average of H_{W}(T,TØh), i.e. H_{W}(ΔT) and H_{W}(R,RØh), i.e. H_{W}(R,RR)
   H_{W}=w_{ΔT}H_{W}(ΔT)+w_{(R,RR)}H_{W}(R,RR)

### Example 5

### Two Channel—Heparin Concentration in Plasma

The embodiment of the two channel heparin concentration method to measure heparin concentrations in plasma and based on a Sonoclot Analyzer is described in the following step by step procedures. This embodiment has the further advantage of being able to be optimized for actual hospital patient populations. Two separate procedures are required: a first procedure to calibrate an instrument for calculating a heparin concentration, and a second procedure to measure the heparin concentration of a sample containing an unknown concentration of heparin. The Sonoclot ACT result is used as the T and the Sonoclot CR result is used as the R.

Heparin Concentration Calibration for Test Reagent
18. Collect both native and citrated whole blood samples from a hospital patient that requires heparin therapy
19. Run the native heparin sample on a Sonoclot Analyzer following manufacturer's instructions on two Sonoclot Analyzer tests: a P2-1 and a P2-2. The Sonoclot ACT run on the P2-1 is T. The Sonoclot ACT run on the P2-2 is TØh. The Sonoclot CR run on the P2-1 is R; the Sonoclot CR run on the P2-2 is RØh.
20. With the citrated whole blood sample, run an anti-Xa heparin concentration assay.
21. Repeat this data collection process on at least 100 patient samples. Include patients across the range of heparin concentrations of clinical interest as well as patients prior to receiving heparin therapy. This will result in a dataset of results and corresponding heparin concentrations in plasma.
22. Calculate the linear and 2nd order regressions
   R (H(aXa))
   ΔT (H(aXa))
   RR (H(aXa))
23. Calculate the regression inverses—these are calibration equations
   H_{P}(R)
   H_{P}(ΔT)
   H_{P}(RR)
24. Using the H(aXa) results, separate a subset with a size of at least 20 samples with the lowest heparin concentrations. From this data subset calculate the mean of the H(aXa) values, μₘᵢₙ
25. From the subset found in Step 21, calculate the standard deviations of the residuals for the heparin concentration estimates and establish the standard error points:
   [μₘᵢₙ, σH_{R@H=μmin}]
   [μₘᵢₙ, σH_{ΔT@H=μmin}]
   [μₘᵢₙ, σH_{RR@H=μmin}]
26. Using the H_{P}(aXa) results, separate a subset with a size of at least 20 samples with the highest heparin concentrations.
27. From the subset found in Step 23, calculate the standard deviations of the residuals for the heparin concentration estimates and establish the standard error points:
   [μₘᵢₙ, σH_{R@H=μmin}]
   [μₘᵢₙ, σH_{ΔT@H=μmin}]
   [μₘᵢₙ, σH_{RR@H=μmin}]
28. Calculate σH_{R@H=min}, standard deviation for the residuals, H_{P}(R)−H(aXa), for low heparin concentration samples
29. Calculate σH_{P@H=max}, standard deviation for the residuals, H_{P}(R)−H(aXa), for high heparin concentration samples
30. Calculate σH_{ΔT@H=min}, standard deviation for the residuals, H_{P}(ΔT)−H(aXa), for low heparin concentration samples
31. Calculate σH_{ΔT@H=max}, standard deviation for the residuals, H_{P}(ΔT)−H(aXa), for high heparin concentration samples
32. Calculate σH_{RR@H=min}, standard deviation for the residuals, H_{P}(RR)−H(aXa), for low heparin concentration samples
33. Calculate σH_{RR@H=max}, standard deviation for the residuals, H_{P}(RR)−H(aXa), for high heparin concentration samples
34. Calculate standard error calibration equations from standard deviation points:
   σH_{P}(R), a linear equation of the standard error across the range of heparin concentrations based on [μₘᵢₙ, σH_{R@H=μmin}] and [μₘₐₓ, σH_{R@H=μmax}]
   σH_{P}(ΔT), a linear equation of the standard error across the range of heparin concentrations based on [μₘᵢₙ, σH_{ΔT@H=μmin}] and [μₘₐₓ, σH_{ΔT@H=μmax}]
   σH_{P}(RR), a linear equation of the standard error across the range of heparin concentrations based on [μₘᵢₙ, σH_{RR@H=μmin}] and [μₘₐₓ, σH_{RR@H=μmax}]

Heparin Concentration Measurement
35. Collect native whole blood sample that may contain heparin
36. Run a Sonoclot Analysis using both a P2-1 and P2-2 activated test.
37. The Sonoclot Analyzer calculates three heparin concentration estimates
   H_{P}(R)
   H_{P}(ΔT)
   H_{P}(RR)
38. The Sonoclot Analyzer calculates variances for each estimate:
   σH_{P}²(R)
   σH_{P}²(ΔT)
   σH_{P}²(RR)
39. The Sonoclot Analyzer combines H_{P}(R) and H_{P}(RR) into a single estimate, H_{P}(R,RR), by calculating a variance weighted average of H_{P}(R) and H_{P}(RR):
   w_{R}=1/σH_{P}²(R)/(1/σH_{P}²(R)+1/σH_{P}²(RR))
   w_{RR}=1/σH_{P}²(RR)/(1/σH_{P}²(R)+1/σH_{P}²(RR))
40. The Sonoclot Analyzer calculates H_{P}(R,RR) and σH_{P}²(R,RR):
   H_{P}(R,RR)=w_{R}H_{P}(R)+w_{RR}H_{P}(RR)
   σH_{P}²(R,RR)=w_{R}σH_{P}²(R)+w_{RR}σH_{P}²(RR)
41. The Sonoclot Analyzer calculates weights for final heparin concentration measurement:
   w_{ΔT}=1/σH_{P}²(ΔT)/(1/σH_{P}²(ΔT)+1/σH_{P}²(R,RR))
   w_{R,RR}=1/σH_{P}²(R,RR)/(1/σH_{P}²(ΔT)+1/σH_{P}²(R,RR))
42. The Sonoclot Analyzer calculates the final heparin concentration measurement as a variance weighted average of H_{W}(T,TØh), i.e. H_{W}(ΔT) and H_{W}(R,RØh), i.e. H_{W}(R,RR)
   H_{W}=w_{ΔT}H_{P}(ΔT)+w_{(R,RR)}H_{P}(R,RR)

### Example 6

### Heparin Response Imbalance

The individual heparin concentration estimates are shown to provide an improved heparin concentration estimate. Another use of these individual heparin concentration estimates is a diagnostic test for abnormal heparin response which can occur if certain coagulation factor deficiencies are present. In this use, a difference or normalized difference or ratio between the heparin concentration estimates rather than a weighted mean of the estimates may be the quantitative result for clinical use. For example, if H(T) and H(R) are close in value, then the sample performed similarly to the normal samples tested during calibration. However, if H(T) and H(R) are not in close agreement, then the blood sample produced inconsistent results. These inconsistent results may be used to identify an underlying abnormal response to heparin within the patient. Accordingly, in this example the heparin parameter of interest is indicative of an abnormal response to heparin.

Some patients are resistant to heparin. However, identifying this resistance is difficult and patients are typically just treated with additional heparin or in more severe situations with fresh frozen plasma to supplement any coagulation factor deficiencies. Comparing [H(T) and H(R)] or [H(T,TØh) and H(R,RØh)] results to each other provides a useful means to identify patients with heparin associated coagulopathies and quantify the severity of the coagulopathy. Additionally, performing this type of analysis on populations that do not encounter heparin management complications to patient populations that do encounter heparin management complications is useful in establishing clinical guidelines to identify patients at greater risk for heparin management complications.

When the heparin concentration estimates H(T) and H(R) or H(T,TØh) and H(R,RØh) are investigated on the prototype dataset using normalized relationship like (H(T)−H(R))/(H(T)+H(R)), the results are reviewed to evaluate if the relationship may be useful. Low heparin concentrations are discarded because of high variance. For heparin concentrations greater than 0.2 IU/mL whole blood, results showed consistency for individual donors. Many donor samples are always positive or always negative across all tested heparin levels. The plotted data shows a very normal distribution. The data shows the consistency and distribution characteristics useful in tests to differentiate abnormals from a patient population. Further identifying useful Normal ranges is achieved using larger datasets.

### Example 7

### Alternate Embodiment

The heparin concentration estimate improves when additional test results are included that correct for patient to patient variability of data when no heparin is present. This baseline data corrects for offset errors in heparin concentration estimates. A further improvement in precision is achieved by correcting for patient to patient variability in heparin sensitivity. This is accomplished by running an additional test that includes a preloaded known amount of heparin within the test. For example, the blood sample can divided into two aliquots with one of the aliquots including a known amount of heparin. Or, the sample is divided into three aliquots with one aliquot having any heparin neutralized, a second having an added known amount of heparin, and a third run with an unaltered sample with an unknown amount of heparin. Such analysis produces additional results that would be able to be calibrated into additional heparin concentration calibration equations and associated standard error equations. The resulting additional heparin concentration estimates can added to the weighted average final heparin concentration result.

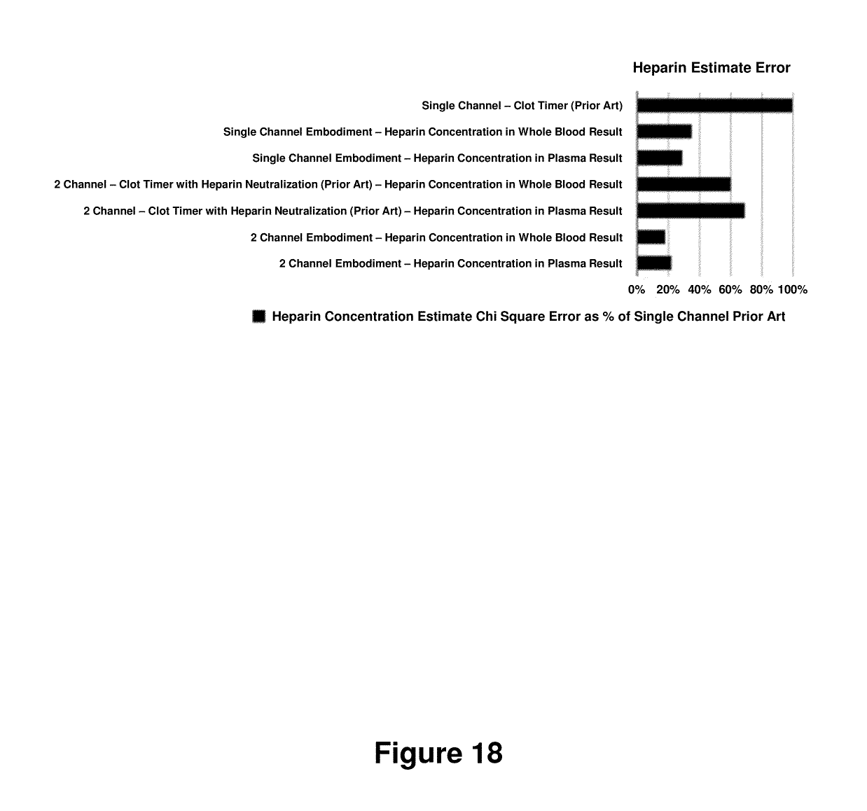

ADVANTAGES: The advantage of this heparin concentration determination method is the unique combination of precision, convenience, and overall value. Precision performance for the heparin concentration method configured as either a single channel or 2 channel instrument is summarized in the Chi Square bar chart of FIG. 18. The example dataset shows that the heparin concentration determination method reduces Chi Square in comparison to prior art for a singled channel by 65% and 71% for whole blood or plasma respectively.

The multiple linear regression model mentioned in a publication but never implemented into an instrument only achieved 53% and 51% reduction in Chi Square for whole blood or plasma (Babski et al. 2012). Further, the multiple linear regression model performance performs far poorer than the heparin concentration determination method as heparin concentration ranges increase. The dataset presented herein extends only from 0 to 0.8 IU/mL whole blood. In other clinical applications, the dataset extends from 0 to over 4 IU/mL. A multiple linear regression analysis loses precision as measurement variances and non-linearity deviations from a linear model spread throughout the data-space. The heparin concentration determination method described herein incorporates non-linear modeling and compensates for measurement variance, allowing this method to be used across wider heparin concentrations.

A two channel embodiment incorporating heparin neutralization differential testing reduces Chi Square in comparison to prior art for a singled channel instrument by 82% and 78% for whole blood or plasma respectively while prior art two channel testing only achieved 40% and 31% Chi Square reduction.

Improved convenience is obtained by reporting test results in useful units. The heparin concentration determination method can be used for reporting heparin concentrations in either whole blood or plasma rather than the current results generated by Clot Timers which are reported as a unit of time and not an actual heparin concentration.

The heparin concentration determination method also can report an estimate of the test result variance or standard error since this data is available within the instrument using the heparin concentration estimate equations and their associated standard error estimates.

The description covers use for native whole blood applications as would be convenient for point of care devices, but the method is compatible with plasma or citrated whole blood samples with appropriate calibration for each type of blood sample being analyzed.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when an element or step is claimed, it should be understood that elements or methods known in the prior art, including certain elements or methods disclosed in the references disclosed herein (particularly in referenced patent documents), are not intended to be included in the claim.

One skilled in the art readily appreciates that the present invention is well adapted to carry out the objects and obtain the ends and advantages mentioned, as well as those inherent in the present invention. The methods, components, materials and dimensions described herein as currently representative of preferred embodiments are provided as examples and are not intended as limitations on the scope of the invention. Changes therein and other uses which are encompassed within the spirit of the invention will occur to those skilled in the art, are included within the scope of the claims.

Although the description herein contains certain specific information and examples, these should not be construed as limiting the scope of the invention, but as merely providing illustrations of some of the embodiments of the invention. Thus, additional embodiments are within the scope of the invention and within the following claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. Definitions provided herein are to clarify their specific use in the context of the invention.

Examples of documents that may be relevant include the following, which are specifically incorporated by reference to the extent not inconsistent herewith:

U.S. Patents

| Patent Number | Issue Date | Patentee |
|---|---|---|
| 4,067,777 | Jan. 10, 1978 | Innerfield, deceased, et al |
| 4,795,703 | Jan. 3, 1989 | Folkman et al |
| 5,702,912 | Dec. 20, 1997 | Hemker et al |
| 5,705,396 | Jan. 6, 1998 | Fickenscher et al |
| 6,489,289 | Dec. 3, 2002 | Nörtersheuser et al |
| 7,247,488 | Jul. 24, 2007 | Ghai et al |
| 7,575,886 | Aug. 18, 2009 | Venkataraman et al |
| 7,699,966 | Apr. 20, 2010 | Qin et al |
| 7,977,106 | Jul. 12, 2011 | Widrig Opalsky et al |
| 8,008,086 | Aug. 30, 2011 | Cohen et al |

U.S. Patent Application Publications

| Publication Nr. | Publ. Date | Applicant |
|---|---|---|
| 20070098593 | May 3, 2007 | Ericson et al |
| 20100105091 | Apr. 29, 2010 | Giesen et al |
| 20100273738 | Oct. 28, 2010 | Valcke et al |
| 20110301120 | Dec. 8, 2011 | Nowakowska et al |
| 20120009686 | Jan. 12, 2012 | Yamamoto et al |

### Nonpatent Literature Documents

D. M. Babski et al., Sonoclot Evaluation of Single- and Multiple-Dose Subcutaneous Unfractionated Heparin Therapy in Healthy Adult Dogs. J Vet Intern Med, Volume 26, Issue 3, 2012, pp 631-638.

### Tables of Terms

TABLE 1
Terms to define blood clotting

| | |
|---|---|
| Activated | A Clot Timer test used to monitor heparin |
| Clotting | |
| Time (ACT) | |
| APTT | A Clot Timer test used to monitor heparin |
| anti-Xa | A laboratory test to measure heparin concentration in |
| | plasma: considered the “Gold Standard” for precision |
| Clot | The phase of the Clotting Model when Clot Integrity |
| Formation | increases |
| Phase | |
| Formation | A quantitative result that characterizes the rate of |
| Rate (R) | Clot Integrity formation |
| Clot | A quantitative assessment of the clot. Prior to any clot |
| Integrity | formation, Clot Integrity is zero. Clot Integrity |
| | increases during the Clot Formation Phase. Weak clots |
| | have lower clot integrity than strong clots. |
| Clot Timer | An instrument capable of measuring the time a coagulation |
| | test progresses until a clot is detected. This result |
| | approximates the Coagulation Reaction Time |
| Reaction | The time from when a clotting test begins until Clot |
| Time (T) | Integrity begins to increase. Also, the time period of |
| | the Coagulation Reaction Phase |
| Global | An instrument capable of measuring Clot Integrity. Any |
| Hemostasis | such device can be used to calculate estimates of both T |
| Analyzer | and R results |
| Coagulation | The phase of the Two Phase Clotting Model prior to any |
| Reaction | clot formation |
| Phase | |
| Two Phase | A simplified model that characterizes clot formation as |
| Clotting | a two phase model consisting of a Coagulation Reaction |
| Model | Phase and a Clot Formation Phase |

TABLE 2
Terms used in Single Channel Embodiment

| | |
|---|---|
| h | Heparin concentration in whole blood |
| H(aXa) | Heparin concentration in plasma as measured by |
| | anti-Xa laboratory test |
| T(h) | Linear regression of T results at known heparin |
| | concentrations in whole blood |
| T(H(aXa)) | Linear regression of T results at measured anti-Xa |
| | heparin concentrations in plasma |
| ΔT(h) | Linear regression of ΔT results at known heparin |
| | concentrations in whole blood |
| ΔT(H(aXa)) | Linear regression of ΔT results at measured anti-Xa |
| | heparin concentrations in plasma |
| R(h) | Higher order regression of R results at known heparin |
| | concentrations in whole blood |
| R(H(aXa)) | Higher order regression of R results at measured anti-Xa |
| | heparin concentrations in plasma |
| RR(h) | Higher order regression of RR results at known heparin |
| | concentrations in whole blood |
| RR(H(aXa)) | Higher order regression of RR results at measured anti-Xa |
| | heparin concentrations in plasma |
| H_{P}(T) | Heparin concentration in plasma estimate from T result |
| H_{W}(T) | Heparin concentration in whole blood estimate from T |
| | result |
| H_{P}(R) | Heparin concentration in plasma estimate from R results |
| H_{W}(R) | Heparin concentration in whole blood estimate from R |
| | results |
| H_{P}(T, R) | Weighted average heparin concentration in plasma estimate |
| | using H_{P}(T) and H_{P}(R) |
| H_{W}(T, R) | Weighted average heparin concentration in whole blood |
| | estimate using H_{W}(T) and H_{W}(R) |
| HRI_{P}(T, R) | Heparin Response Imbalance in plasma derived from |
| | H_{P}(T) and H_{P}(R) |
| HRI_{W}(T, R) | Heparin Response Imbalance in whole blood derived |
| | from H_{W}(T) and H_{W}(R) |

TABLE 3
Terms used in Two Channel Embodiment

| | |
|---|---|
| Th | T Without Heparin |
| Rh | R Without Heparin |
| H_{P}(T, Th) | Heparin concentration in plasma estimate from |
| | T and Th results |
| H_{W}(T, Th) | Heparin concentration in whole blood estimate |
| | from T and Th results |
| H_{P}(R, Rh) | Heparin concentration in plasma estimate from |
| | R and Rh results |
| H_{W}(R, Rh) | Heparin concentration in whole blood estimate |
| | from R and Rh results |
| H_{P}(ΔT) | Heparin concentration estimate, H_{P}(T, Th), with |
| | ΔT being the relationship between T and Th |
| H_{W}(ΔT) | Heparin concentration estimate H_{W}(T, Th), with |
| | ΔT being the relationship between T and Th |
| H_{P}(RR) | Heparin concentration estimate H_{P}(R, Rh), with |
| | RR being the relationship between R and Rh |
| H_{W}(RR) | Heparin concentration estimate H_{W}(R, Rh), with |
| | RR being the relationship between R and Rh |
| H_{P}(T, R, Th, Rh) | Heparin concentration in plasma estimate from |
| | T, R, Th, and Rh results |
| H_{W}(T, R, Th, Rh) | Heparin concentration in whole blood estimate |
| | from T, R, Th, and Rh results |
| H_{P}(T, R, ΔT, RR) | same as H_{P}(T, R, Th, Rh) except using |
| | the alternate terms, ΔT and RR |
| H_{W}(T, R, ΔT, RR) | same as H_{W}(T, R, Th, Rh) except using |
| | the alternate terms, ΔT and RR |
| HRI_{P}(T, R, Th, Rh) | Heparin Response Imbalance in plasma derived |
| | from H_{P}(T, Th) and H_{P}(R, Rh) |
| HRI_{W}(T, R, Th, Rh) | Heparin Response Imbalance in whole blood |
| | derived from H_{W}(T, Th) and H_{W}(R, Rh) |
| Clot Formation Rate | The ratio: Rh/R |
| Ratio (RR) | |
| ΔT | The difference: T − Th |

TABLE 4
Terms used in Calibration Equations

| | |
|---|---|
| σH_{P}(T) | Standard error estimate of H_{P}(T) |
| σH_{P}(R) | Standard error estimate of H_{P}(R) |
| σH_{P}(ΔT) | Standard error estimate of H_{P}(ΔT) |
| σH_{P}(RR) | Standard error estimate of H_{P}(RR) |
| σH_{W}(T) | Standard error estimate of H_{W}(T) |
| σH_{W}(R) | Standard error estimate of H_{W}(R) |
| σH_{W}(ΔT) | Standard error estimate of H_{W}(ΔT) |
| σH_{W}(RR) | Standard error estimate of H_{W}(RR) |
| σH_{W}(R, R) | Standard error estimate of H_{W}(R, RR) |
| σH_{P: T@H=LOW} | standard deviation of H_{P}(T) − Hₚ(aXa) |
| | for low heparin concentration samples |
| σH_{W: T@H=0} | standard deviation of H_{W}(T) − h at heparin = 0 |
| σH_{P: T@H=HIGH} | standard deviation of H_{P}(T) − Hₚ(aXa) |
| | for high heparin concentration samples |
| σH_{W: T@H=MAX} | standard deviation of H_{W}(T) − h at heparin = |
| | maximum heparin level |
| σH_{P: R@H=LOW} | standard deviation of H_{P}(R) − Hₚ(aXa) |
| | for low heparin concentration samples |
| σH_{W: R@H=0} | standard deviation of H_{W}(R) − h at heparin = 0 |
| σH_{P: R@H=HIGH} | standard deviation of H_{P}(R) − Hₚ(aXa) |
| | for high heparin concentration samples |
| σH_{W: R@H=MAX} | standard deviation of H_{W}(R) − h at heparin = |
| | maximum heparin level |
| σH_{P: ΔT@H=LOW} | standard deviation of H_{P}(ΔT) − Hₚ(aXa) |
| | for low heparin concentration samples |
| σH_{W: ΔT@H=0} | standard deviation of H_{W}(ΔT) − h at heparin = 0 |
| σH_{P: ΔT@H=HIGH} | standard deviation of H_{P}(ΔT) − Hₚ(aXa) |
| | for high heparin concentration samples |
| σH_{W: ΔT@H=MAX} | standard deviation of H_{W}(ΔT) − h at heparin = |
| | maximum heparin level |
| σH_{P: RR@H=LOW} | standard deviation of H_{P}(RR) for low heparin |
| | concentration samples |
| σH_{W: RR@H=0} | standard deviation of H_{W}(RR) − h at heparin = 0 |
| σH_{P: RR@H=HIGH} | standard deviation of H_{P}(RR) for high heparin |
| | concentration samples |
| σH_{W: RR@H=MAX} | standard deviation of H_{W}(RR) − h at heparin = |
| | maximum heparin level |
| μₘᵢₙ | The mean of H(aXa) for low heparin concentration |
| | samples |
| μₘₐₓ | The mean of H(aXa) for high heparin concentration |
| | samples |
| w_{T} | Weight of either H_{W}(T) or H_{P}(T) estimate: used in |
| | calculating weighted average of multiple heparin |
| | concentration estimates |
| w_{R} | Weight of either H_{W}(R) or H_{P}(R) estimate |
| w_{ΔT} | Weight of either H_{W}(ΔT) or H_{P}(ΔT) estimate |
| w_{RR} | Weight of either H_{W}(RR) or H_{P}(RR) estimate |

TABLE 5
Terms related to Sonoclot ™ Coagulation
& Platelet Function Analyzer (Sienco, Inc.)

| | |
|---|---|
| ACT | Sonoclot terminology for a T result |
| CR | Clot Rate: Sonoclot terminology for a R result |
| P2-1 | Sonoclot Analyzer test containing an APTT activation reagent |
| P2-2 | Sonoclot Analyzer test consisting of a P2-1 with the additional |
| | reagent heparinase to neutralize heparin |

We claim:

1. A method for determining a heparin parameter in a fluid sample that may include heparin, said method comprising the steps of:
providing said fluid sample;
measuring a first parameter from said fluid sample, wherein said first parameter varies with heparin concentration in said fluid sample;
measuring a second parameter from said fluid sample, wherein said second parameter varies with heparin concentration in said fluid sample;
calculating from said first parameter a first intermediate result and from said second parameter a second intermediate result; and
combining said first and said second intermediate results to determine said heparin parameter, wherein said heparin parameter is heparin concentration or heparin response imbalance.

2. The method of claim 1 wherein said heparin concentration is a measure of heparin that is selected from the group consisting of:
unfractionated heparin; and
low molecular weight heparin.

3. The method of claim 1 wherein said fluid sample is selected from the group consisting of:
native whole blood;
citrated whole blood;
citrated plasma; and
citrated platelet rich plasma.

4. The method of claim 1 wherein one of said first or second parameter characterizes a reaction phase prior to clot formation.

5. The method of claim 4, wherein said reaction phase prior to clot formation is one or more of:
prothrombin time;
International Normalized Ratio;
partial thromboplastin time;
activated partial thromboplastin time;
activated clotting time;
Thromboelastography R;
Thromboelastography R+k;
Sonoclot ACT;
Sonoclot Onset Time;
Rotem RT;
Rotem CT;
Rotem CFT;
Thromboscope Lag time; or
an optical property obtained from an optical transmission or chromogenic plasma coagulation analyzer.

6. The method of claim 1 wherein one of said first or second parameter characterizes a clot formation phase.

7. The method of claim 6 wherein said clot formation phase is one or more of:
Thromboelastography k;
Thromboelastography α;
Thromboelastography MA;
Thromboelastography T;
Thromboelastography A30 or A60;
Sonoclot Clot Rate;
Rotem MCF;
Rotem MCF-t;
Rotem CFT;
Rotem α;
Rotem A5, A10;
Thromboscope Time to Peak;
Thromboscope Time to Peak—Thromboscope Lag Time;
Thromboscope Peak;
Thromboscope ETP;
Thromboscope slope of calibrated automated thrombogram;
Thromboscope maximum acceleration of calibrated automated thrombogram; or
a parameter derived from a clot curve developed from an optical transmission or chromogenic plasma coagulation analyzer.

8. The method of claim 1, wherein said first and second parameters are determined by an instrument that generates a measurable parameter of said fluid sample to determine said first and second parameters using:
viscosity measurement;
elastic measurement;
optical transmission measurement;
optical diffusion measurement; or
ultrasonic measurement.

9. The method of claim 1 wherein said first and second intermediate results are each heparin concentration estimates.

10. The method of claim 1 wherein said heparin parameter is heparin concentration.

11. The method of claim 1, further comprising:
calculating an estimated variance for each of said first and second intermediate results;
wherein said intermediate results and estimated variances are calculated using an estimation equation and an estimate variance equation derived from a dataset of parameter results from a collection of blood samples with each blood sample of said collection having a known heparin concentration.

12. The method of claim 11 wherein said heparin parameter is calculated by combining said first and second intermediate results using a weighted average.

13. The method of claim 11 wherein a weight is assigned to said first and second intermediate result and said weight is the inverse of said estimated variance of said intermediate result.

14. A method of claim 1 wherein said heparin concentration is reported in units selected from the group consisting of:
International Units (IU) per mL whole blood; and
International Units (IU) per mL plasma.

15. A method of claim 1 wherein said intermediate results are heparin concentration estimates and said heparin parameter is a numerical comparison of the individual heparin concentration estimates to assess normal or abnormal response to heparin.

16. The method of claim 1, further comprising dividing said fluid sample into a first and a second fluid sample, wherein said first fluid sample does not contain active heparin, and performing said steps on each of the first and second samples.

17. A method for determining a heparin parameter in a fluid that may include heparin, said method comprising steps of:
providing a first fluid sample and a second fluid sample from a donor, wherein said first fluid sample does not contain active heparin;
measuring a first parameter from each of said first and second fluid samples, wherein said first parameter varies with heparin concentration in said fluid sample;
measuring a second parameter from each of said first and second fluid samples, wherein said second parameter varies with heparin concentration in said fluid sample;
calculating from said first parameter a first intermediate result and from said second parameter a second intermediate result for said first fluid sample;
calculating from said first parameter a second intermediate result and from said second parameter a second intermediate result for said second fluid sample; and
combining said first and second intermediate results from said first fluid sample and said second sample to determine said heparin parameter, wherein said heparin parameter is heparin concentration or heparin response imbalance.

18. The method of claim 17, wherein said heparin concentration is a measure of heparin that is selected from the group consisting of:
unfractionated heparin; and
low molecular weight heparin.

19. The method of claim 17, wherein said fluid sample is selected from the group consisting of:
native whole blood;
citrated whole blood;
citrated plasma; and
citrated platelet rich plasma.

20. The method of claim 17, wherein said first fluid sample is extracted from said fluid prior to adding any heparin.

21. The method of claim 17, wherein said providing said first fluid sample step comprises: neutralizing or removing heparin from said first fluid sample.

22. The method of claim 21, wherein said neutralizing step comprises applying a sufficient amount of heparinase to neutralize at least a portion of the heparin in said first fluid sample.

23. The method of claim 17, wherein one of said first or second parameter characterizes a reaction phase prior to clot formation.

24. The method of claim 23, wherein said reaction phase prior to clot formation is one or more of:
prothrombin time;
International Normalized Ratio;
partial thromboplastin time;
activated partial thromboplastin time;
Thromboelastography R;
Thromboelastography R+k;
Sonoclot ACT;
Sonoclot Onset Time;
Rotem RT;
Rotem CT;
Rotem CFT;
Thromboscope Lag time; or
an optical property obtained from an optical transmission or chromogenic plasma coagulation analyzer.

25. The method of claim 17, wherein one of said first or second parameter characterizes a clot formation phase.

26. The method of claim 25, wherein said clot formation phase is one or more of:
Thromboelastography k;
Thromboelastography α;
Thromboelastography MA;
Thromboelastography T;
Thromboelastography A30 or A60;
Sonoclot Clot Rate;
Rotem MCF;
Rotem MCF-t;
Rotem CFT;
Rotem α;
Rotem A5, A10, . . . ;
Thromboscope Time to Peak;
Thromboscope Time to Peak—Thromboscope Lag Time;
Thromboscope Peak;
Thromboscope ETP;
Thromboscope slope of calibrated automated thrombogram;
Thromboscope maximum acceleration of calibrated automated thrombogram;
a parameter derived from a clot curve developed from an optical transmission or chromogenic plasma coagulation analyzer.

27. The method of claim 17, wherein said first and second parameters are determined by an instrument that generates a physical parameter of said fluid sample to determine said first and second parameters using:
viscosity measurement;
elastic measurement;
optical transmission measurement;
optical diffusion measurement; or
ultrasonic measurement.

28. The method of claim 17 wherein said first and second intermediate results are each heparin concentration estimates.

29. The method of claim 17 wherein said heparin parameter is heparin concentration.

30. The method of claim 17, further comprising:
calculating an estimated variance for each of said first and second intermediate results for each of said first and second fluid samples,
wherein said intermediate results and estimated variances are calculated using an estimation equation derived from a dataset of parameter results from a collection of blood samples with each blood sample of said collection having a known heparin concentration;

31. The method of claim 17 wherein said heparin parameter is calculated by combining said first and second intermediate results into a weighted average for each of said first fluid sample and second fluid sample.

32. The method of claim 31, wherein a weight is assigned to said first and second intermediate result for each of said first and second fluid sample and said weight is the inverse of said estimated variance of said intermediate result.

33. A method of claim 17, wherein said heparin concentration is reported in units selected from the group consisting of:
International Units (IU) per mL whole blood; and
International Units (IU) per mL plasma.

34. A method of claim 17, wherein said intermediate results are heparin concentration estimates and said heparin parameter is a numerical comparison of the individual heparin concentration estimates to assess normal or abnormal response to heparin.

35. The method of claim 17, further comprising determining heparin concentration from said second fluid sample and identifying the donor of said first and second fluid samples as having an abnormal heparin response.
